(19) United States
(12) Patent Application Publication
(10) Pub. No.: US 2006/0115895 A1
(43) Pub. Date: Jun. 1, 2006

(54) Fish disease models and uses thereof

(76) Inventors: Paul Goldsmith, Cambridge (GB); Angeleen Louise Fleming, Cambridge (GB); Andrew John McGlashan Richards, Cambridge (GB); Derek Thomas Edward Jones, Cambridge (GB); William Anthony Harris, Cambridge (GB)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/537,258

(22) PCT Filed: Dec. 1, 2003
(86) PCT No.: PCT/GB03/05239

§ 371 Date: Nov. 3, 2005

(30) Foreign Application Priority Data
Dec. 2, 2002 (GB) 0228093.1

(51) Int. Cl. C12N 5/00; C12N 5/02

(52) U.S. Cl. 435/325

(57) ABSTRACT

Generation and use of disease models, for instance in assays to identify and investigate genes and substances involved in disease and disease treatment, identification and use of drug targets. In particular, the present invention relates to generation and use of disease models in fish such as zebrafish. Gradable phenotypes may be generated in fish, allowing for degree of correction or alteration of an activity or effect of a treatment, gene or mutation to be assayed. Competing stimuli may be applied to fish in screens assaying for ability to affect an activity or effect of a treatment, gene or mutation. Example include any combination of an addictive drug, a visual stimulus, an audible startle stimulus, a temperature gradient, an electric shock, an aversive compound, a threatening shape or a dark area.

The present invention relates to generation and use of disease models, for instance in assays to identify and investigate genes and substances involved in disease and disease treatment, identification and use of drug targets. In particular, the present invention relates to generation and use of disease models in fish such as zebrafish. In some preferred embodiments, gradable phenotypes are generated in fish, allowing for degree of correction or alteration of an activity or effect of a treatment, gene or mutation to be assayed. In some preferred embodiments, competing stimuli are applied to fish in screens assaying for ability to affect an activity or effect of a treatment, gene or mutation. For example any combination of an addictive drug, a visual stimulus, an audible startle stimulus, a temperature gradient, an electric shock, an aversive compound, a threatening shape (e.g. model of a predatory animal) or a dark area may be used. Other preferred embodiments are disclosed herein.

Drug discovery is currently limited by the ability to know whether inhibition of a particular gene, biological pathway or a combination of several genes/pathways will have a desirable effect on a particular disease state in vivo. The present invention provides strategies to overcome these problems, including methods to develop the appropriate disease models, how to subsequently screen these models, then how to translate this into human therapeutics. By combining the appropriate steps in a particular way the overall aim is achieved. The invention thus lies not only in the nature of particular individual steps, but also in the particular way these individual steps are combined together. The nature of certain steps places constraints on other steps. Thus, an additional part of the invention lies in the recognition of these constraints and the application of particular strategies, both in that particular step, and in the other steps, with the aim of overcoming these constraints.

The optimal model system for the in vivo practice of the enclosed disclosure is a fish, especially a zebrafish. The zebrafish is an organism which combines many of the advantages of mammalian and invertebrate model systems. It is a vertebrate and thus more relevant to models of human disease than Drosophila or other invertebrates, but unlike other vertebrate models it can readily be used to perform genetic screens.

The inventors have appreciated that zebrafish offer the unique combination of invertebrate scalability and vertebrate modelling capabilities. They develop rapidly, with the basic body plan already having been laid out within 24 hours of fertilization. Moreover, their ex-utero development within a transparent capsule allows the easy in vivo visualisation of internal organs through a dissecting microscope. Many disease states can be modelled within the first week of life, at which time the embryos are only a few millimetres long and capable of living in 100 ul of fluid. This permits analysis of individual embryos in multi-channel format, such as 96 well plate format. This is particularly useful for drug screening, with many chemicals being arranged in 96 well plate format.

Alternatively, a population of fish in a petri dish or a tank may be employed. A population of fish may be treated together, and may be tested together, e.g. via addition of one or more or a combination of test substances to the water.

The zebrafish has a short maturation period of two to three months and is highly fecund, with a single pair of adults capable of producing 100 to 200 offspring per week. Both embryos and adults are small, embryos being a few mm and adults 2-3 cm long. They are cheap and easy to maintain. The ability to generate large numbers of offspring in a small place offers the potential of large scalability.

A further advantage of zebrafish is the fact they live in water. This makes administration of candidate chemicals easy. Moreover, the inventors have discovered that zebrafish are also DMSO (dimethyl sulphoxide) tolerant. This is important as many drugs are dissolved in DMSO. The inventors have established that zebrafish can tolerate 1% DMSO. Thus, a candidate drug or other test substance may be dissolved in DMSO and administered to zebrafish by adding to the fish water to give a final concentration of DMSO of at least up to 1%. This is employed in various preferred aspects and embodiments of the present invention.

Zebrafish and other fish also readily absorb chemicals. The effective concentration of chemicals in the water often equates to the effective plasma concentration in mammals.

Thus, zebrafish enable the entire biological pathway of a vertebrate to be screened in a high-throughput fashion.

It is possible to introduce random mutations into the zebrafish genome, for example with the use of chemical mutagenesis (Solnica-Krezel et al. Genetics 1994, 136(4): 1401-20). The publication of the results of the first large scale mutagenesis screens was in 1996 by the Nüsslein-Volhard and Driever groups [Driever, 1996; Haffter, 1996]. They were able to isolate over 2000 mutants affecting nearly every aspect of embryogenesis during the first few days of development. The original 1996 screens analyzed 2746 strains of embryos. Only those with obvious morphological abnormalities visible under the dissecting microscope were kept. Thus the fish selected on the basis that they might have a visual problem were those with alterations in eye shape, size or pigmentation. 49 mutants were isolated this way. Such screening methods inevitably introduce bias into mutant selection. It is also likely that only a small fraction of the total number of possible mutants were identified, as most of the loci isolated were only represented by a single allele.

Various strategies have been devised to increase the sensitivity of primary mutagenesis screens. In the original screens, microscopic examination was used to detect degeneration of particular anatomical areas of interest, by observing a decrease in the size of the structure or altered optical properties using Nomarski optics. Now, groups are creating transgenic lines expressing GFP under the control of promoters, thus expressed in subtypes of cells of interest to speed up screens, similar to that previously used in other animals (e.g. WO01/12667). Mutant fish can then be screened for loss of fluorescence and thus abnormal development or degeneration of a particular cell. An example is the transgenic line expressing GFP in rod photoreceptors, by placing the rod opsin promoter upstream of eGFP (Kennedy et al., J Biol Chem (2001) 276, 14037-14043). It is also possible to make transgenic fish carrying exogenous genes. Zebrafish expressing a heterologous Ikaros protein have been used to model haematopoiesis and lymphoproliferative disorders (WO 01/40273).

WO99/42606 concerns a method of screening an agent for an angiogenesis activity or cell death activity or toxic activity, comprising administering the agent to a teleost (e.g. zebrafish, medaka, Giant rerio or puffer fish), and detecting a response in the teleost indicating angiogenesis activity or an effect on cell death activity or toxic activity in at least one tissue or organ of the teleost.

WO01/12667 describes use of a transgene to drive marker expression in the eye. The organism may be fish. It suggests making a transgenic animal (which it says may be a fish) by a method comprising introducing a genetic construct for expression of a marker sufficient to visually detect the marker in photoreceptive cells or organ and selecting for transgenesis by visually detecting the marker in a photoreceptive cell or organ.

WO01/51604 (Exelixis) is concerned with providing sensitizer genes such as a tumor gene or an oncogene in a non-human animal (for which zebrafish are mentioned as a passing, hypothetical possibility) in cells where expression is non-lethal. It is proposed to detect changes and compare on mutation or other treatment. The aim is identification of “interactor genes” that, when mutated, specifically kill or reduce the size of target tissue (subject to the sensitizer gene), by visually observing the change in appearance of the tissue. For this reason they chose the drosophila ommatidia, as death of the cells of the ommatidia give a rough-looking appearance to the fly eye when viewed under a microscope.

WO98/56902 discloses use of transgenic fish, including zebrafish, and methods of crossing fish strains, including strains with mutations, the aim being to identify genes that affect expression of fish genes.

Scott C. Baraban, Peter A. Castro and Herwig Baier have disclosed identification of seizure resistant zebrafish mutants as a model of epilepsy. For induction of seizures, zebrafish larvae were exposed to a common proconvulsant agent (pentylenetetrazole, PTZ) and the fish were observed to undergo three distinct stages of seizure-like behavior, as described previously (Baraban et al. 2001; Epilepsia 42:3.017). Twelve families that carried putative seizure-resistance mutations were identified.

In contrast to the above disclosures, the present invention provides particular methods which enable a fish, such as a zebrafish, to be used efficiently in a wide variety of situations to discover therapeutics relevant to human disease. Provided by the present invention are fish (e.g. zebrafish) disease models and disease modelling methods which are not only representative of the underlying disease, but are also particularly amenable for use in subsequent screening. This allows in turn to the identification of a human therapeutic. The invention is generally applicable to any of a variety of diseases and disorders, and a range of examples is specifically set out herein.

A particularly important aspect in selecting drug targets or compounds for possible development is assessing the likely degree of effect on the disease state, rather than just an assessment of whether it has a detectable effect or not. It is valuable to be able to quantitate the potential therapeutic effect at the earliest opportunity and rank this against other candidate compounds and targets. Only those compounds or targets with a large enough effect to translate into a clinically useful effect can then be selected for further development. Equally, only those compounds or targets with a large enough effect to be detected in the clinical trial with the degree of power which will subsequently be run can then be selected for further development.

The present invention provides means, specifically fish such as zebrafish, for use in methods of screening for and identifying a gene which, when mutated, alters the activity or effect of a disease gene and thus a disease phenotype. A secondary gene of which mutation affects activity or effect of a primary disease gene is termed an “interactor” gene and if it reduces activity or effect of a primary gene is termed a “suppressor” gene. If it increases activity of effect of a primary gene it is termed an “enhancer” gene. Interactor genes, including suppressor and enhancer genes, represent targets for drugs to treat the disease. A particular advantage of interactor gene screens is that the interactor gene may be part of an unexpected pathway, but will still be identified in such a screen. Additionally, as drugs commonly bind to and antagonise their targets, a drug which binds to the protein encoded by the wild type interactor gene, may have a similar beneficial effect on disease phenotype.

In addition to zebrafish, other fish such as fugu, goldfish, medaka and giant rerio are amenable to manipulation, mutation and study, and use in aspects and embodiments of the present invention as disclosed herein.

The present invention is concerned in various aspects and embodiments with a method of screening for a substance or gene that affects activity or effect of a second gene (e.g. expression or function of a gene or encoded protein), or activity or effect of a treatment, on behaviour or physiology of a fish, the method comprising:
providing fish transgenic for the second gene or subject to said treatment, as model fish for screening;
mutating said model-fish to provide mutated fish or treating said model fish with a test substance to provide treated fish;
comparing behaviour or physiology of mutated fish or treated fish with model fish in order to identify any mutated fish or treated fish with altered behaviour or physiology compared with model fish;
thereby to identify a test substance that affects activity or effect of the second gene or activity or effect of said treatment, or by identifying a genetic difference between model fish and mutated fish with such altered behaviour or physiology to identify a gene that affects activity or effect of the second gene or activity or effect of said treatment.

The invention in various aspects and embodiments provides various modifications and developments of such a method.

Thus, for example, in one aspect the present invention provides a method of screening for a substance or gene (termed herein “first gene”) that affects activity or effect of a second gene, or activity or effect of a treatment, on a fish, the method comprising:
providing, as model fish for screening, (i) fish transgenic for the second gene, wherein the second gene is under regulatory control of a specific promoter and expression of the second gene within the fish affects an aspect of behaviour or physiology of the fish, or (ii) fish subject to said treatment, wherein the treatment affects an aspect of behaviour or physiology of the fish;
mutating said model fish to provide mutated fish or treating said model fish with a test substance to provide treated fish;
comparing an aspect of behaviour or physiology of mutated fish or treated fish with that of model fish in order to identify any mutated fish or treated fish with altered behaviour or physiology compared with model fish;
thereby to identify a test substance that affects activity or effect of the second gene or activity or effect of said treatment, or, by identifying a genetic difference between model fish and mutated fish with such altered behaviour or physiology to identify a first gene that affects activity or effect of the second gene or activity or effect of said treatment.

Optionally, such a method further comprises screening for and preferably identifying or obtaining a chemical that interacts with the protein encoded by the wild-type first gene, e.g. for use as a therapeutic in the treatment of human disease.

A specific promoter may be used, and a specific promoter is generally tissue-specific and/or inducible or derepressible. A preferred promoter allows the disease state to be recapitulated, whilst also allowing all subsequent steps in the screening procedure to be carried out. Allowing expression of the disease in entirety under the control of its natural promoter, as described with previously disclosed prior art, may not permit these subsequent steps to be performed, and, in those circumstances in which they could be performed, they may not offer the equivalent ability to identify a therapeutic relevant to the treatment of human disease. An inducible promoter may be responsive to an applied stimulus, while a promoter that can be derepressed is active upon removal of a repressor. In some preferred aspects of the present invention the specific promoter may not be eye-specific in the fish and/or the behaviour or physiology of the fish that is compared may not be vision, although in other aspects and embodiments eye-specific expression may be employed and/or assessment and comparison of vision. In various preferred embodiments of the invention, the specific promoter is selected from the group consisting of nicotinic acetylcholine receptor beta3, rhodopsin, Flil, keratin8, islet-1, Type II cytokeratin, muscle creatine kinase, alpha actin, acidic ribosomal phosphoprotein P0, Beta actin, Pdx1, insulin, alpha1 tubulin, transducin, CRX, phosphodiesterase, ath5, brn3c, alphaB crystallin, tyrosine hydroxylase, dopamine decarboxylase, tyrosinase, GATA-2 and GATA-1 promoters. Tissues in which a harmful gene may be expressed include, but are not restricted to: neurons, subsets of neurons (including motor neurons), components of the visual system (e.g. photoreceptors, lens, ganglion cells), muscle, components of the auditory system, the skin, the swim bladder, the pancreas, the haematopoetic system (including specific haematopoetic subtypes), the vasculature and the heart.

Promoters which have already been shown to direct expression to specific cell types in zebrafish include:

Neuronal cells: nicotinic acetylcholine receptor beta3 (nAChRbeta3) promoter [Tokuoka, 2002]

Photoreceptors: rhodopsin promoter [Perkins, 2002].

Blood vessels: Flil promoter [Lawson, 2002].

Stratified epithelium: keratin8 promoter [Gong, 2002].

Motor neurons: islet-1 promoter [Higashijima, 2000].

Skin: Type II cytokeratin promoter [Ju, 1999].

Muscle: Muscle creatine kinase promoter [Ju, 1999]; Alpha actin promoter [Higashijima, 1997].

General expression: acidic ribosomal phosphoprotein P0 (arp) gene [Ju, 1999]; Beta actin promoter [Higashijima, 1997).

Pancreas: Pdx1 and insulin promoters [Milewski, 1998; Huang, 2001].

Neuronal progenitors: alpha1 tubulin promoter [Goldman, 2001]; GATA-2 promoter [Meng, 1997].

Haematopoetic cells: GATA-1 promoter [Long, 1997; Meng, 1999].

Lens: alphaB crystallin [Posner, 1999]

In preferred embodiments, the aspect of behaviour or physiology that is to be determined for model and mutated and/or treated fish is gradable, i.e. can be quantitated.

In some preferred embodiments, level of skin pigmentation is determined for fish, and can be compared as between model fish and mutated and/or treated fish. Zebrafish adapt their skin colour to the ambient light intensity by varying their concentration and distribution of melanin. In a dark environment melanin granules become widely distributed within melanophores, star-shaped black pigment cells, thus causing the fish to look blacker. In contrast, in a light environment the melanosomes aggregate. The stimulus for this adaptation depends on a retino-hypothalamic projection, the hypothalamus then inducing pituitary secretion of either a hormone causing melanin aggregation or one causing melanin dispersal [Odenthal, 1996]. By creating dysfunction specifically in a component of this pathway, such as through the selective expression of a disease-causing gene, it is possible to get a very easy and rapid read-out of the level of dysfunction through analysis of pigmentation. For example, by expressing the disease-causing gene in that subset of ganglion cells which project to the hypothalamus to mediate the background adaptation response, it is possible to model neuronal degeneration in the absence of any generalised neuronal dysfunction and with the read-out of pigmentation level. Alternatively, the pathological process could be confined to the epithelial cells, again with a read-out of pigmentation level, although other markers, such as degree of expression of a fluorescent marker within the epithelial cells may also be used.

In the circumstance that extent of pigmentation is used as the read-out, this may be quantitated by comparing the test fish with a wild-type fish. Pigment granules, in particular the melanin granules or melanosomes, contained within melanophores, absorb light. Thus by illuminating the fish with light of standard intensity and then measuring the amount of light transmitted through to the far side of the fish container, a difference in light absorption and therefore pigmentation level may be detected between test and control fish. A suitable detector would be the Dynatech™ microplate reader, a photometer designed to measure the light absorbance of samples in a microtitre plates. This is automatable, high-throughput and gradable, thus allowing small differences in pigmentation to be detected. This is important as the suppression or enhancement of a disease may be slight. The smaller the difference that can be detected and the smaller the amount of time taken to detect this, the greater the chance of identifying an interactor or chemical which is altering the baseline phenotype.

In other preferred embodiments, auditory function is determined, and can be compared as between model fish and mutated and/or treated fish. Especially when combined with other steps, this allows for detection of small changes in baseline in a high throughput fashion. Fish exhibit an acoustic startle in which a sudden auditory stimulus is detected by otolithic sensory hair cells, in turn leading to firing of the eight nerve. A related vibratory reflex, resulting from a sudden vibrational stimulus, is mediated through the lateral line. These stimuli then activate Mauthner cells, which in turn activate motor neurons. The result is muscle contraction, evidenced by a reflexive swim.

A sharp tap to the side of a container of the fish, e.g. a Petri dish, may be used to elicit the startle response. An auditory stimulus may be standardised by dropping a small weight from a predefined distance onto the side of the container, e.g. Petri dish, or other consistently repeatable mechanical stimulus. Sound may be applied by means of an acoustic stimulus, e.g. a loud speaker. For example, a loud speaker may be attached to a container of the fish, e.g. to the base or side of a Petri dish, and a noise played through the loudspeaker. Such a noise need only be brief.

A change in movement amplitude may be quite subtle. In order to quantitate changes in movement amplitude the following steps may be performed, in accordance with aspects and embodiments of the present invention:
1. Distance that a weight falls onto a container such as a Petri dish may be altered, with an observed threshold height for a response in a control situation compared with that in a test situation.
2. Amplitude of a sound played through a loudspeaker may be gradually changed to determine a threshold to observe a response in both a control and test situation.
3. Amplitude of response of fish under examination may be quantitated by recording the response on a CCD camera and then measuring the distance moved following the stimulus.

Extent of neuronal activity may be accurately measured by determining the degree of fluorescence in embryos which have been injected with 4-6 nl of 0.5-1% calcium green-1 dextran (Molecular Probes) at the 1 to 4 cell stage (Nicolson). This acts as a quantitative marker of neuronal activity. Similar activity sensitive markers may also be used. A morphological assessment of the integrity of the hair cells is also possible using a fluoroscein-phalloidin stain (Molecular Probes), followed by a comparison of structure and fluorescence intensity between control and test fish. A similar assessment of the neuromasts is also possible, should the disease process be engineered to lead to neuromast degeneration. In this case DAPSEI staining (Molecular Probes) would be appropriate.

Vision may be compared, in combination with one or more additional features in various aspects and embodiments of the present invention as disclosed herein.

Where the behaviour and/or physiological response is seizure, especially if seizure induce by pentylenetetrazole, PTZ, and especially if in zebrafish, this may be employed in aspects and embodiments of the present invention in combination with one or more additional features as disclosed herein. Pilocarpine, kainic acid or high dose penicillin are examples of proconvulsants that may be used in aspects and embodiments of the present invention.

Where a treatment is applied to fish initially to affect an aspect of behaviour or physiology and provide model fish that can then be subject to mutation to provide mutated fish or subject to treating with a test substance to provide treated fish, in certain embodiments the treatment is not by application solely of pentylenetetrazole, PTZ.

The present invention provides in various aspects and embodiments for applying to or exposing fish such as zebrafish to two opposing stimuli. This allows for increased sensitivity and gradability of an assay and allows teasing apart of small differences in response tendency. Opposing stimuli may be selected for example from any combination of light stimulation, optomotor stimuli, temperature, whether with discrete changes or a temperature gradient, food, aversive chemicals or drugs, attractive or additive chemicals, physical aversion such as electric shock and a threatening shape.

As a preferred embodiment, zebrafish are habituated to an additive substance, which may be nicotine. This may be through the addition of the substance to fish water, e.g. for a period of 3 days, before the water is then replaced with fresh water. The natural response is one of desire to receive more of the additive substance, e.g. further nicotine. The fish will now swim preferentially towards a focal source of the addictive substance, such as supplied through a wick to one end of the tank. An opposing stimulus may be applied, e.g. passage of black and white stripes in the case of the optomotor response, a temperature gradient or an aversive stimulus, and strength of this opposing stimulus may be altered. By adjusting the stimuli appropriately, fish which are very addicted will still swim towards the addictive substance such as nicotine in the face of the opposing stimulus. In contrast, fish which have had their addictive tendency ameliorated as a result of a test substance or genetic alteration will now be drawn away from the addictive stimuli. By adjusting the amplitude of this other counter-attraction tending to draw the fish away from the addictive stimulus, the extent of the addiction can be reliably teased out and quantitatively assessed. In a further specific embodiment, a source of the addictive substance such as nicotine may be contained in ice, or at a cold end of the tank. Fish prefer to avoid cold and thus the temperature which they are prepared to swim into in order to reach the nicotine source provides an accurate gradable assessment of their degree of addiction. By these means, together with a mutagenesis or chemical screen, genes and drugs may be identified which alter the addiction response, highly desirable for the treatment of human drug addiction. This is given as an example and does not imply any limit the scope of such a technique.

The present invention in certain aspects and embodiments provides for screening for and preferably identifying or obtaining a substance that provides a synergistic combination with another substance, or for screening for and preferably identifying or obtaining two or more substances that together provide an additive or synergistic combination. Clinical benefit is often derived from synergistic combinations of drugs. Use of an in vivo system in accordance with the present invention allows for identification of such synergistic combinations.

Thus, in certain embodiments the invention comprises generation of a model fish, as disclosed, treating model fish with two or more substances, at least one of which is a test substance, and comparing the effect of the two or more substances in combination (whether simultaneously or sequentially applied) on an aspect of behaviour or physiology with the effect of either or both of the two or more substances when applied individually or alone. Either all (or both) of the substances applied may each be a test substance, or one of the substances may be a drug known to have a beneficial effect in the disease that is the subject of the model, or at least an effect in the model fish.

The invention thus provides for screening for and preferably identifying or obtaining a substance that provides an additive effect to a known drug or a synergistic effect with the known drug. It also provides for screening for and preferably identifying or obtaining a combination of two or more substances that provide a synergistic effect, compared with the effect of the two substances when employed individually or alone.

Add-on therapies are useful because it is difficult to conduct clinical trials in which an existing drug is withdrawn from a patient and replaced with a new drug. The patient is deprived of a drug which has at least got some proven efficacy and some confidence in its side-effect profile. Additionally, the patient will be vulnerable to their disease during the phases of withdrawal of the existing drug and build up of the test drug. For example in the case of epilepsy, the patient would be at risk of fits during this period. Thus, although the new drug may be more effective in monotherapy than the existing drug, most clinicians and patients would not want to take the risk of switching therapy and having a fit, with the social and medical implications, including the loss of a driving licence in many countries. It is thus much more desirable to trial an add-on therapy.

The present invention provides strategies for the identification of combination therapies, add-on therapies and synergistic combinations in vivo. Continuing with the example of epilepsy by way of illustration, in embodiments of this aspect of the present invention a known drug may be administered to the fish, for example sodium valproate. Epilepsy is then induced in the fish. Additional test substances, such as candidate chemicals, or genetic mutation may then be applied to the fish and assessed for their anti-epileptogenic effect. If this effect is greater than any of the chemicals/drugs/mutations alone, then a clinically useful effect has been identified, such as an augmentation, combination or synergistic effect. The following drugs are examples that in combinations of two or more may have some synergistic action together: sodium valproate, ethosuximide, phenyloin, carbamazepine, lamotrigine, gabapentin, phenobarbitone, diazepam, clobazam, tiagabine and levetericatem. Any combination of these and/or other drugs or test substances may be arrayed in every possible combination and tested for synergistic efficacy. Appropriate doses vary from 1 nM to 100 mM, e.g. 0.1 μM to 10 mM, with 100 uM being a reasonable starting concentration to assess. This will pick up beneficial effects, with for example, diazepam showing therapeutic effects in monotherapy at 35 uM concentration.

Furthermore, the invention provides the ability to avoid the development of a compound or target that would otherwise be toxic when co-administered with an existing therapy, or would decrease the beneficial effects of an existing therapy. (e.g. it is useful to know as early as possible that the new anti-epileptic in development is not going to lessen the effect of sodium valproate, lamotrigine or carbamazepine when eventually prescribed in the clinic, given that many patients will be taking and remaining on one of these drugs).

In a detailed example of an embodiment of the invention, wild-type fish of tup background are reared to day seven. Seizures are then induced in the fish through addition of a stock of pentylenetetrazole solution in order to achieve a concentration of 10 uM in the fish water. Diazepam is added to the fish to a concentration of 20 uM, either on day 6, concurrent with the administration of the pentylenetetrazole, or 2 hours subsequent to the administration of the pentylenetetrazole. In addition to the diazepam and pentylenetetrazole, an additional test substance or test substances, are added to achieve a final concentration of 100 uM. Controls include wild type fish with:
1. only dilutant added
2. only pentylene tetrazole added
3. only pentylene tetrazole and diazepam added
4. only test substance added
5. only pentylene tetrazole and test substance added

The extent of seizures is then assayed through visual observation of the presence or absence of seizure activity and the duration of each seizure after 6 hours. If the additional test substance decreases the seizure activity in comparison to controls 2-5, then it can be concluded that the test substance may be a useful add-on therapy to diazepam therapy in the clinical situation.

Various timings of administration and assessment, various doses and various drugs may be used. The above provides just one example.

In addition to a test substance, the fish may be a mutated fish rather than a wild-type fish. It is then possible to assay for interacting effects, either beneficial synergistic effects, or deleterious effects, of the mutation plus the test substances. Alternatively, the analysis may be of the known therapeutic agent and the genetic mutation in order to discover either a new drug target of benefit in combination with the known drug, or a genetic marker of use in predicting which patients are most likely to benefit (or not benefit) from prescription of the known drug.

In another embodiment, a combination of potential anti-inflammatory agents is administered to a model of an inflammatory disease, which may be generated through addition of a pro-inflammatory agent to the fish water, through expression of a pro-inflammatory gene, or knock-down of an anti-inflammatory gene, to assess whether the combination is more effective than either of the individual agents. For example, there are a variety of anti-inflammatory agents, either in clinical trials or currently prescribed. For the sake of this example, assume there are 11 drugs to be tested. It is possible that the various drugs act at different pinch points in biological pathways and that by judicious co-prescribing, an optimal combination may be found that is better than any drug alone, whilst with no worse a side effect profile. It would be very difficult to do clinical trials, or indeed mammalian studies to determine the optimum combination. The present invention allows this.

For example in an array of wells, drug 2 is added to every well in both row 2 (“B”) and column 2, drug 3 to every well in row 3 (“C”) and column 3, and so on. Row 1 (“A”) and column 1 are control wells in which solvent only is added. Each well also contains fish with inflammation induced. The degree of inflammation is then assessed, ideally in a graded high-throughput fashion. In this example the untreated fish in well A1 have a maximum disease severity of 10, all others having a disease severity of 5-9, except for C6 and F3 which have grade 2 disease severity. Thus drugs 3 and 6 have a synergistic effect.

This was tested experimentally: Compound DL 494 when tested alone at 40 ng/ml in an in vivo model of inflammation, showed no detectable response. Similarly, compound DL567, when tested alone at 40 ng/ml in an in vivo model of inflammation, showed no detectable response. A mix of the 2 enantiomers was tested at 20 ng/ml of each enantiomer (40 ng/ml overall of compound) resulting in full rescue of the inflammation phenotype.

The present invention also provides for screening for and preferably identifying or obtaining a substance that ameliorates one or more side effects of an active substance, e.g. a therapeutically active substance. There are many drugs which have been discontinued in clinical trials, or are marketed but infrequently prescribed, not because they are not therapeutically effective, but because their side-effect profile is limiting. The side-effects may be relatively benign, but significant to the patient, such as weight gain (e.g carbamazepine), or of potentially fatal consequence, such as inducing a cardiomyopathy (e.g. doxorubicin). It is desirable to allow the administration of such drugs, with proven beneficial effects, through the co-administration of an additional agent to improve the side-effect profile. An example of this is the co-administration of H2 antagonists (e.g. ranitidine) or proton pump inhibitors (e.g. omeprazole) to decrease the risk of gastric ulceration in patients who require NSAIDS (e.g. ibuprofen) for pain relief or aspirin as an anti-platelet aggregatory agent.

In accordance with the present invention, such agents are screened for in fish in which administration of the active substance induces a side-effect or other phenotype reflective or indicative of a side-effect. Thus in embodiments of the invention, an active agent is administered to fish and the side-effect of other phenotype is assessed for such fish when subjected to one or more test substances. This does not require a priori knowledge of action of the co-administered agent. In other embodiments, agents that achieve the desired therapeutic effect with a reduction of side-effects can be screened for and preferably identified or obtained by means of assessment of disease phenotype and side-effect phenotype. As with other aspects and embodiments of the present invention, this may involve co-administration of a primary compound together with either a battery of candidate substances, or together with randomly induced genetic mutation. With the latter approach, i.e. mutation, subsequent steps are needed to identify the appropriate co-therapeutic following identification of fish with a mutation that provides an ameliorative effect.

A diverse library of drug-like compounds, such as the LOPAC library (Sigma) may be used, or the Chembridge PHARMACOphore diverse combinatorial library. Other targeted libraries against particular targets classes may be used, such as ion channel libraries or G protein libraries.

Still further provided by the present invention is a method of identifying mutations, genotypes, allelic variations, haplotypes and genetic profiles associated with responsiveness to a therapeutic. There is an increasing move towards targeted prescribing, whereby the choice of therapeutic is influenced by genotyping the patient. Particular polymorphisms have been found to predict both the therapeutic effectiveness of a compound, and also the likelihood of suffering certain side effects. Such rationalised prescribing is cost-effective. It also makes clinical trials easier to run, as likely responders can be targeted, thus necessitating a smaller sample size to achieve statistical significance. However, for the moment, most drugs, both already prescribed or in development, do not have an appropriate test.

The present invention provides for assessing the effectiveness of various medications in combination with random genetic mutations to identify those mutations which either enhance or decrease the therapeutic effectiveness and/or alter the side effect profile. This allows for identification of genes, polymorphisms, mutations, alleles and haplotypes associated with a particular response to a drug or other treatment, enabling development of appropriate genetic assays in humans to permit rationalised prescribing.

For example, the effectiveness of diazepam in reducing seizures is assayed in an array of fish with a variety of mutations. It is found that diazepam at a concentration of 30 uM has an anti-seizure effect of grade 3 in wild type fish. However, in the presence of mutation x, the anti-seizure effectiveness is increased above grade 3, whilst in the presence of mutation y, the anti-seizure effectiveness is decreased below grade 3. Thus diazepam should be preferentially prescribed to patients with a mutation or polymorphism equivalent to mutation x in the homologous gene, and preferentially avoided in patients with a mutation or polymorphism equivalent to mutation y in the homologous gene.

In a further embodiment, rather than target the prescribing of a beneficial agent, or improve the efficacy of an already beneficial agent, the invention may be used to reduce the side effects of an agent which otherwise might not be prescribed because of its negative side effect profile. In this situation the deleterious side effect is assayed, with an improvement of this deleterious side effect being examined for through the result of an additional chemical or interactor gene.

For example, mitoxantrone has useful clinical effects in the treatment of multiple sclerosis [van de Wyngaert, 2001], but its use is severely curtailed on account of the cardiac side effect profile [Ghalie, 2002]. Fish with random mutations or bathed in a test substance are exposed to mitoxantrone and examined for a change in cardiac function compared to baseline. An improvement in function from baseline treatment with mitoxantrone alone, leads to the identification of the interactor gene, or chemical and subsequently therapeutic, to be co-prescribed with mitoxantrone in multiple sclerosis patients.

Embodiments of the present invention as disclosed herein may employ expression of the shibire or tetanus toxin genes in order to achieve either a temperature controlled inactivation of a specific circuit of interest, whilst maintaining neuronal structure, or cell death of neurons of interest, respectively.

A method of the invention may comprise mutating model fish transgenic for the second gene to provide mutated fish and identifying a first gene that affects activity or effect of the second gene.

A method of the invention may comprise treating with a test substance model fish transgenic for the second gene to provide treated fish and identifying a test substance that affects activity or effect of the second gene.

A method of the invention may comprise mutating model fish subject to said treatment to provide mutated fish and identifying a first gene that affects activity or effect of said treatment.

A method of the invention may comprise treating with a test substance model fish subject to said treatment to provide treated fish and identifying a test substance that affects activity or effect of said treatment.

A method of the invention may comprise identifying a first gene that lessens activity or effect of the second gene.

The second gene may be a disease gene.

A method of the invention may comprise identifying a first gene that enhances or increases activity or effect of the second gene.

A method of the invention may comprise identifying a test substance that lessens activity or effect of the second gene or said treatment.

The second gene may be a disease gene.

A method of the invention may comprise identifying a test substance that enhances activity or effect of the second gene or said treatment.

In advantageous embodiments of the present invention, a disease model is generated by application of a chemical or physical treatment, rather than by genetic mutation, although use of genetic mutation is involved in other embodiments of the present invention as disclosed herein. Chemical or physical induction of a disease state allows all fish in a population or test sample to have the disease induced at the same time in a controlled fashion, and then tested for genetic or chemical rescue. It can be used to overcome difficulties of disease lethality, and allows for rapid model generation. In preferred embodiments, the phenotype is gradable, which is advantageous in rescue screening.

The creation of a genetically altered line is time-consuming. Additionally, if the mutation affects survival or breeding capacity, maintenance of the genetically altered line may be compromised. Furthermore, if the mutation has early developmental effects, in addition to its disease-causing effects, this may compromise the appearance of the disease phenotype. Finally, when considering subsequent screening for rescue, as only a proportion of any clutch derived from breeding of the parent carriers will manifest the disease, it is necessary to either screen populations of fish rather than individual fish, or invent additional screening steps to allow the identification of the carrier fish (see elsewhere herein). This is because if a fish appears normal, one would not otherwise know whether this was because it was a wild-type, or a mutant fish which had been rescued from disease.

The induction of disease by chemical or physical methods can be used to overcome many of these issues. Chemicals are known to induce disease in mammals with phenotypic equivalence to human disease. For example, intraperitoneal injection of streptozotocin into mice induces pancreatic beta cell death after a period of 2 weeks [Hassan, 2001], phenotypically resembling type 1 diabetes mellitus. Chemicals have also been shown to cause specific defects in fish as part of a screen to dissect the genetics of development [Peterson, 2000]. It is by combining this disease induction with suitable phenotypic screening methods and rescue strategies (which may involve treatment with a test substance and/or mutation) that agents relevant to the rescue of human disease can be identified.

In certain embodiments, effects on vision may be determined.

For example, thyroxine is known to switch photoreceptor fate towards cones and away from rods [Kelley, 1995], whilst rods do not contribute significantly to vision until day 10-14 [Saszik, 1999]. Carbimazole decreases thyroxine levels [Taurog, 1976] and thus, when administered to zebrafish embryos, could lead to cone degeneration and blindness.

Other potential methods of modelling ophthalmic disease include the administration of the following drugs, known to cause retinal or optic nerve dysfunction as a side effect: methanol [Eells, 2000]; quinine and choloroquine [Calissendorff, 1976]; ethambutol [Heng, 1999]. The ensuing visual disturbance is then amenable to a highly specific, gradable and high-throughput screening assay in accordance with the present invention such as the use of the optomotor assay. The administration of physical agents may also be used to induce a disease state, such as a retinal degeneration, in an analogous way to the addition of a chemical, such as the exposure to bright lights to induce retinal degeneration (Vihtelic, 2000].

Visual function of a fish such as a zebrafish may for example be assayed one of the following ways:

(1) Zebrafish change colour to blend in with background. In a dark environment the fish turn blacker and in a bright environment lighter. Blind fish perceive themselves as being in the dark and thus turn black. This provides a very rapid visual screen—the black fish are blind.

(2) Zebrafish or other fish optokinetic response can be assayed by passing stripes in front of the eyes. As in human subjects, the resultant reflexive eye movements cannot be suppressed. Thus passage of stripes in front of fish such as zebrafish and the assessment of the presence or absence of optokinetic response provides another assay for visual function. A related visual assay involves the optomotor response. The sophistication of stimuli may be built up to allow a detailed, graded assessment of fish, e.g. zebrafish, visual function. Moreover, the assessment mechanisms allow for the testing of larger numbers of fish in a short period of time.

A moving grating or a movie, e.g. presented as a computer-animated display on a screen, elicits innate optomotor behavior in zebrafish larvae; they swim in the direction of perceived motion (Orger et al. Nat Neurosci 2000 November 3(11):1128-33). Zebrafish larvae innately begin responding to moving stimuli shortly after hatching. This is advantageous in a screen of the present invention as disclosed herein, since it allows for rapid determination of the effect of mutation on a model zebrafish. Other fish show similar responses.

Visual function of zebrafish may be determined by considering their colour or background pigmentation, alteration of which is indicative of blindness as discussed. Visual function may be determined by means of observation of response to a pattern of e.g. of stripes passed in front of the eyes of the fish. In a preferred embodiment, black fish expected to be blind and lighter-coloured fish expected to have visual function are segregated on the basis of their colour, prior to determination of the degree or extent of visual function by means of a visual assay.

Assaying for visual function of a fish such as a zebrafish may employ observation of a change in background pigmentation, of visual startle, of optokinetic response and/or optomotor response. The latter two both typically involve passage of a pattern, e.g. horizontal stripes, passed. In an optokinetic assay, fish may be immobilized in 3% methylcellulose and the pattern passed in front of the eyes. In an optomotor assay, fish are free swimming in one or more long, narrow channels, and the pattern is passed along the length of the channels. Fish swim in the perceived direction of motion of the stipes and thus collect at one end of the channel, unless they are blind in which case they are distributed randomly.

A typical powerful stimulus is a 100% contrast square wave filling the entire visual field, and a stripe width of 36 degrees moving at 4 Hz. Limits of vision are identified in Orger et al., supra, although for partially blind fish the limits are less. Degree of visual dysfunction can be determined by decreasing the contrast and/or stripe width of a pattern until the fish no longer respond. Other aspects of visual dysfunction may be analysed by varying the colour of the stripes or other pattern.

The pattern may be stripes or wavy lines moving across the field of vision. Other non-Fourier or second order stimuli may be employed, e.g. as described in Orger et al., supra.

Fish may be treated with a substance in a number of ways, either as treatment to create a primary phenotype in which fish are affected in an aspect of behaviour or physiology, or in treating fish with a test substance in the course of a screen for a test substance able to alter an effect of a primary treatment or mutation on a primary phenotype. Fish may be contacted with a test substance, it may be touched or rubbed on their surface or injected into them. A test substance may be added to water in which they are, or in the case of a protein, produced in the cell via expression of the appropriate coding sequence.

In further preferred embodiments, the disease model in fish is generated by means of expression of a transgene that induces an effect on an aspect of behaviour and/or physiology of the fish, a measurable and preferably gradable phenotype.

The expression construct may encode antisense RNA. Thus a series of lines of zebrafish expressing various antisense RNAs may be generated and crossed to, or expressed in, the disease situation. IF an altered phenotype is seen, the rescuing gene can be readily identified by PCR of the antisense sequence.

This may also be combined with the Gal4/UAS system with bidrectional promoters in the cases of diseases modelled using the gal4/UAS system.

The promoter used to control expression, which may be tissue-specific expression may be inducible, which may facilitate establishment and/or screening of a fish line.

Tissue-specific and/or inducible expression can be used to overcome difficulties with lethality, and allows for provision of a gradable phenotype to screen (e.g. skin pigmentation, auditory response, swimming).

In particular embodiments the disease gene when expressed results in a disease phenotype in a dominant fashion. Some embodiments of the invention involves placing the gene under the control of a promoter, rather than its own natural promoter, to avoid lethality, the promoter for example being inducible and/or tissue-specific. The disease is then only manifest under conditions in which the promoter is induced and/or in tissues in which the promoter is active. This can be used to allow fish to reach an age of viability and fecundity. For example, using an inducible promoter allows for the disease process to be switched off before the fish die and not switched on until any pleiotropic actions have terminated.

A preferred inducible promoter for use in embodiments of the present invention is a heat-shock promoter, such as HSP70 [Yeh, 2000]. Use of such a promoter allows for induction of a disease state in a controlled fashion by means of alteration of temperature. Fish are amenable to prolonged expression from a heat-shock promoter, which may be necessary before a phenotype is detectable, as temperature of water within which fish reside can be easily adjusted and maintained. The inventors have observed that zebrafish can survive at a wide variety of temperatures. Only minor changes in temperature are necessary to activate heat shock promoters [D'Avino, 1999].

A disease gene to be employed in an embodiment of the present invention may be any gene in wild-type or mutant form which, when expressed in a fish such as zebrafish results in abnormal development, dysfunction or degeneration of tissue or cell function.

In further preferred embodiments of the present invention, a Gal4/UAS system is used. GAL4 encodes a yeast (Saccharomyces cerevisiae) protein of 881 amino acids, that regulates genes induced by galactose. It does so by directly binding to four related 17 bp sites, together defining an Upstream Activating Sequences (UAS) element, analogous to a multicellular eukaryotes enhancer element. GAL4 can function in a wide variety of organisms to activate transcription from the UAS element. This permits targeted gene expression in a temporal and spatial fashion in vivo. To achieve this, transcription of the responder gene is controlled by presence of the UAS element. To activate their transcription, responder lines are mated to lines expressing GAL4 in a particular tissue of interest, termed the driver. The resulting progeny then express the responder in the desired tissue of interest.

This can be used to overcome difficulties with lethality, allows the same driver lines to be used for several diseases and facilitates creation of mutated lines for subsequent genetic rescue. The gal4/UAS system was originally developed as a means of tissue specific gene expression in Drosophila [Brand, 1993; Brand, 1994). Such a system is now also being used to assess developmental genes in zebrafish [Scheer, 2002].

In this system, a driver line with a GAL4 coding sequence is placed under the control of a tissue specific promoter, such as CRX, a homeobox gene expressed specifically in photoreceptors [Furukawa, 2002], or other, depending on the desire tissue specificity for expression. A responder line is then generated in which the disease-inducing gene is coupled to the UAS promoter. This promoter is driven by GAL4. Thus, when the two lines are crossed together, a proportion of the offspring will carry both constructs. In these fish, the disease-causing gene will be expressed in a tissue-specific fashion.

In preferred individual embodiments, the dominant acting disease gene is selected from the group consisting of huntingtin, alpha-synuclein, presenilin-1, presenilin-2, TNF, SMN, Tau and rhodopsin, either in wild-type or mutant forms. Accession numbers and references for these genes are as follows, and are all incorporated herein by reference:

Huntingtin: accession for wild-type human gene OMIM 143100; Huntington's Disease Collaborative Research Group, Cell 72: 971-983, 1993. PubMed ID: 8458085, Marsh et al. Hum Mol Genet 2000; 9(1):13-25;

Alpha-synuclein: accession for wild-type human gene XM003494; Spillantini et al. Nature 1997; 388(6645):839-40;

Presenilin-1: accession for wild-type human gene AH004968; Sherrington et al. Nature 375 (6534), 754-760 (1995);

Presenilin-2: accession for wild-type human gene NM_{—}000447; Levy-Lahad et al. Science 269 (5226), 973-977 (1995), Levy-Lahad et al. Science 269 (5226), 970-973 (1995), Rogaev et al. Nature 376 (6543), 775-778 (1995), Levy-Lahad et al. Genomics 34 (2), 198-204 (1996);

TNF: accession for wild-type human gene XM_{—}055614; Bitsch et al. Glia. 2000 Feb. 15; 29(4):366-75, Liu et al. Nat Med. 1998 January; 4(1):78-83, Probert et al. Proc Natl Acad Sci USA. 1995 Nov. 21; 92(24):11294-8;

SOD-1: accession for wild-type human gene AY049787; Rosen et al. Nature 1993; 362(6415):59-62, Parkes et al. Nat Genet 1998; 19(2):171-4;

SMN: accession for wild-type human gene XM_{—}041493; Pellizzoni et al. Cell 1998, 95(5):615-24, Miguel-Aliaga et al. FEBS Lett 2000; 486(2):99-102;

Rhodopsin: accession for wild-type human gene U49742, Kaushal et al. Biochemistry 1994; 33(20):6121-8, Li et al. Proc Natl Acad Sci USA 1996; 93(24):14176-81, Colley et al. Proc Natl Acad Sci USA 1995; 92(7):3070-4.

Tau: accession for wild-type human gene NM_{—}173727, Heutink, P. Hum. Mol. Genet. 9 (6), 979-986 (2000); Goedert et al. Proc. Natl. Acad. Sci. U.S.A. 85 (11), 4051-4055 (1988).

Beta amyloid 1-40 or beta amyloid 1-42 from the beta amyloid A4 precursor protein (OMIM *014760).

For example, by coupling the huntingtin gene to a marker, or by assessment of the disease phenotype, such as gradable visual psychophysics, or through genotyping, it is possible to assess those fish which should express the disease. By adding a test substance (or applying any of the other inventions disclosed herein), it is possible to identify a beneficial therapeutic substance. To identify a genetic mutation with a beneficial effect, an additional step may be performed.

This may involve exposing adult male heterozygous of homozygous driver lines to mutagenesis, for example with ENU. These males are then crossed with female responder lines. If the induced mutation has a dominant interactor effect, a deviation from the expected phenotype will result in those fish harbouring both the driver and responder constructs. The inventors term this a Gal4 F1 mutagenesis phenotype interactor screen.

Similarly, in the case of a recessive heterozygous carrier, or a dominant disease causing gene expressed in a tissue specific fashion, mutagenesis of the adult males followed by crossing of these males with carrier females and screening of the F1 progeny for deviation from the expected phenotype, achieves the desired aim of the identification of an interactor gene.

Additionally or alternatively, the coding sequence of a gene, (e.g. a gal4 gene to achieve additional temporal and spatial expression control) may be placed under the control of an inducible promoter, such as a promoter selected from a heat shock promoter, or a tetracycline or hormone inducible system.

The invention provides for manipulation of nucleic acid in order to modify cells of fish such as zebrafish, as disclosed. Nucleic acid of a disease gene to be expressed in fish in accordance with the invention is to be integrated into the chromosome of cells. Integration may be promoted by inclusion of sequences which promote recombination with the genome, in accordance with techniques available in the art. The disease gene may be heterologous to the fish, e.g. may be heterologous to zebrafish (e.g. mammalian, such as human), and may be in wild-type form or in any allelic or mutant form. The disease gene may be a zebrafish or other fish gene, in wild-type or mutated form, e.g. to provide an extracopy of a zebrafish or other fish gene, such as in a mutated disease form.

Nucleic acid sequences encoding the peptides or polypeptides of the present invention may be readily prepared by the skilled person using the information and references contained herein and techniques known in the art (for example, see Sambrook and Russell “Molecular Cloning, A Laboratory Manual”, Third Edition, Cold Spring Harbor Laboratory Press, 2001, and Ausubel et al, Current Protocols in Molecular Biology, John Wiley and Sons, 1992, or later edition thereof). See Detrich et al. (1998) The Zebrafish: Biology. Methods in Cell Biology. Volume 59, and Detrich et al. (1998) The Zebrafish: Genetics and Genomics. Methods in Cell Biology. Volume 60 for techniques of zebrafish maintenance, mutagenesis, transgenesis and mapping.

The desired coding sequence may be incorporated in a construct having one or more control sequences operably linked to the nucleic acid to control its expression. Appropriate regulatory sequences, including promoter sequences, terminator fragments, polyadenylation sequences, enhancer sequences, marker genes and other sequences as appropriate may be included.

Regions responsible for promoter and enhancer activity of a gene known to be expressed in a desirable pattern such as only under certain conditions or in certain tissue, may be isolated by ligating stretches of sequence from upstream of the translation start codon in the gene to a reporter gene. Constructs with deletions in putative promoter and/or enhancer regions are generated and the constructs tested for tissue specific gene expression in transgenic fish, e.g. transgenic zebrafish, fugu, goldfish, medaka and giant rerio.

A selectable marker, for example gene encoding a fluorescent protein such as Green Fluorescent Protein (GFP) may be included to facilitate selection of clones in which the gene construct has inserted into the genome. Where a fluorescent marker is used, embryos may be screened under a fluorescent dissecting microscope. Embryos, or fish into which they grow, may be screened for the presence of a defect resulting from the transgene. In another approach, embryos may be pooled prior to extraction of genomic DNA and analysis of the genomic DNA by PCR and/or restriction enzyme digest. Positive clones may be expanded and developed into breeding fish. These fish may then be bred to produce fish which carry one copy of the gene construct in the germ line. These heterozygous fish may then be bred to produce fish carrying the gene homozygously.

A further aspect provides a method which includes introducing a nucleic acid construct wherein a coding sequence of a desired disease gene is placed under control of a promoter into an embryo cell of a fish, e.g. zebrafish. DNA may be injected directly in vivo into cells of an early embryo. With the establishment of embryonic stem cell culture, other methods generally referred to without limitation as “transformation”, may be employed, for instance selected from any method available in the art, such as using calcium phosphate transfection, DEAE-Dextran, electroporation, liposome-mediated transfection and transduction using retrovirus or other virus. Marker genes such as antibiotic resistance or sensitivity genes may be used in identifying clones containing nucleic acid of interest, as is well known in the art.

In a further aspect, the present invention provides a method of making a fish, such as a zebrafish, useful in or for use in a screen as disclosed herein and discussed further below. Such a method may comprise providing a gene construct wherein a coding sequence of a disease gene is operably linked to a promoter that has the desired inducibility and/or tissue specificity, in the fish, introducing the gene construct into a fish embryo, causing or allowing the gene construct to integrate into the fish embryo genome, and growing the fish embryo into a viable fish.

A viable and reproductive fish, e.g. zebrafish, may mate with one or more other fish, establishing a line of fish, e.g. zebrafish, transgenic for the gene construct comprising the disease gene operably linked to, and under regulatory control of, the promoter. A line of such fish, e.g. zebrafish, is useful in screens as disclosed.

In order to introduce a disease gene into a fish embryo, e.g. a zebrafish embryo, a gene construct is made, using techniques available to those skilled in the art. The construct may be released from a vector by restriction digest, and gel purified, for example by elution in 1×TE (pH8.0) and dilution to a working concentration of 50-100 ug/ml KCl containing a marker dye such as tetramethyl-rhodamine dextran (0.125%). Typically, 1 to 3 nl of this solution may be injected into single celled zebrafish embryos. Several thousand embryos may be injected.

Injected embryos are grown up and then mated with each other or to a non-transgenic wild-type fish. Transmission of the transgene to the subsequent generation is usually mosaic, ranging from 2 to 90%. At least 100 offspring are typically analysed to establish whether the founder fish carriers the transgene.

The injected embryos may be reared and assessed for visual function at intervals (e.g. 2 days) by means of modified optokinetic and optomotor assays, e.g. as discussed further below.

Fish demonstrating a desired phenotype and/or genotype may be grown up and may be mated with wild-type fish. The parents and offspring may be matched and the offspring similarly assessed for phenotype and/or genotype. Those offspring with a particular phenotype, and hence likely germline transmission of an integrated disease gene construct, can be selectively bred. Some of the offspring may be sacrificed for more detailed analysis, e.g. to confirm the nature of the blindness. This analysis may include in situ hybridisation studies using sense and anti-sense probes to the introduced gene to check for expression of the construct in cells of the fish, anatomical assessment such as with plastic sections to check for an effect on tissue or cells, and terminal deoxyuridine nucleotide end labelling (TUNEL) to check for apoptotic cell death in cells.

Families from which fish with the appropriate characteristics came may be maintained through subsequent generations. This maintenance then allows this new mutant strain to be entered into a secondary screen in accordance with further aspects of the invention.

Another aspect of the present invention provides cells of transgenic fish, such as zebrafish, fugu, goldfish, medaka and giant rerio as disclosed, whether isolated cells or cell lines derived from the fish and optionally immortalised using standard techniques.

A gene such as a disease gene sequence (e.g. heterologous to fish, such as heterologous to zebrafish) to be employed in aspects and embodiments of the present invention may employ a wild-type gene or a mutant, variant or derivative sequence may be employed. The sequence may differ from wild-type by a change which is one or more of addition, insertion, deletion and substitution of one or more nucleotides of the sequence shown. Changes to a nucleotide sequence may result in an amino acid change at the protein level, or not, as determined by the genetic code.

It is well known that pharmaceutical research leading to the identification of a new drug may involve the screening of very large numbers of candidate substances, both before and even after a lead compound has been found. This is one factor which makes pharmaceutical research very expensive and time-consuming. Means for assisting in the screening process can have considerable commercial importance and utility. Such means for screening for substances potentially useful in treating or preventing a disorder or disease is provided by fish such as zebrafish according to the present invention. Modifier genes, such as enhancer or suppressor genes identified using the invention, and substances that affect activity of such suppressor genes represent an advance in the fight against disease since they provide basis for design and investigation of therapeutics for in vivo use, as do test substances able to affect activity or effect of a treatment, and substances that affect activity or effect of expression of a disease gene in a fish.

In various further aspects the present invention relates to screening and assay methods and means, and substances identified thereby.

The present inventors have realised that fish such as zebrafish are useful in a secondary suppressor or enhancer screen. A secondary suppressor screen involves introducing one or more mutations into the genome and screening or selecting for negation or suppression or enhancement of the effect of a primary mutation.

The principle can be illustrated by way of example, with reference to a hypothetical gene B of which normal function is to control fish size. If this gene is mutated so that it is underactive (a hypomorphic mutant), smaller fish will ensue. Now take another hypothetical gene S whose normal function is to make fish smaller. If this gene is mutated, such that the gene is also underactive, the fish will be bigger. Thus if a mutation is introduced into the S gene in a fish already harbouring a mutated B gene, the two will cancel each other out and the fish will be normal sized. The mutated S gene suppresses the phenotype of the mutated B gene.

However, there is a problem where gene B is a dominant disease gene, since if this makes the fish non-viable, causes them to die quickly or fail to reproduce, it will not be possible to raise adult fish harbouring a mutation in gene B.

The present invention provides a solution to this problem by restricting expression of the disease gene to one or more tissues or particular conditions, by placing it under the control of a suitable promoter, e.g. tissue specific and/or inducible, or by phenocopying the effect of the mutation at a defined time point. As a result of this spatially and/or temporally restricted expression, the disease process is limited to these specific cells. The fish are viable, can be raised to adulthood and bred, and are thus amenable to use in a secondary suppressor screen.

Although in some embodiments of the present invention, disease model induction is generated by chemical or physical means, in many cases it is not possible to do this. This is particularly the case for example for neurological disease, which in many cases is genetically driven. Often the result of the genetic defect is degeneration of the nervous system (e.g. Alzheimer's, Parkinson's, Motor Neuron Disease, Lewy Body Dementia). There is often a lot of phenotypic variation clinically. Moreover, even with the availability of sophisticated clinical assays for human patients, it can be very hard to measure the extent of disease and alterations in progression. This becomes even more difficult with in vivo screening of an animal model.

One solution to this is to express the disease gene in a specific tissue, malfunction of which gives a more easily screenable, quantifiable and gradable phenotype. The present invention in various aspects and embodiments capitalises on unique features of fish, especially zebrafish, and some examples are here given:

a. Eye Specific Expression.

Use of an eye-specific promoter to generate fish with affected vision and subsequent assessment and comparison of vision of fish may be excluded from certain aspects and embodiments of the present invention but is useful in others, such as the use in conjunction with the gal4/uas system to increase expression control, the use with mutation of male heterozygotes to aid identification of interactor genes, the use in conjunction with add-on or combinatorial chemistry screens, or its use in pharmacogenomic screens.

When employed, an eye-specific promoter for use in the present invention provides for expression of a gene e.g. disease gene in a component or tissue of the eye involved in one or more of: the passage of a photon of light through the eye, the conversion of photon energy into a biological signal, and the transmission of this signal to the visual centres of the brain. Thus, a promoter for use in the present invention may be functional in one or more of any of the following tissues and cells: conjunctiva, cornea, aqueous humour, vitreous humour, lens, retina, optic nerve, any retinal cell, rod photoreceptor, cone photoreceptor, ganglion cell, glial cell, horizontal cell, bipolar cell and amacrine cell.

A gene to be employed in an embodiment of the present invention whereby vision of fish is affected may be any gene in wild-type or mutant form which, when expressed in a fish such as zebrafish, results abnormal development, dysfunction or degeneration of a tissue or cell of the eye (e.g. as set out already above), this affecting visual function of the fish. In some embodiments, the expression of the disease gene in a component of the eye causes failure of development or degeneration of a part of the visual system. In other embodiments, the expression of the disease gene in a component of the eye causes a component of the visual system to malfunction.

Alpha-synuclein (e.g. human) carrying A53T mutation may be expressed in fish retinal ganglion cells, leading to a degeneration of the ganglion cells. Transmission of visual information to the brain is consequently impaired, and thus visual function of the fish adversely affected. Fish with alpha-synuclein expressed in their retinal ganglion cells perform more poorly than normal on visual testing.

Rhodopsin (e.g. human) carrying P347S mutation may be expressed in fish photoreceptors, leading to a degeneration of the photoreceptor cells. Consequently, photons are no longer detected by the retina and thus the vision of the fish is impaired, as determined on visual testing.

In one particular embodiment, a coding sequence of a disease gene to be introduced into the fish, e.g. zebrafish, is placed under regulatory control of the ath5 promoter. Ath5 is the zebrafish eye-specific ortholog of the Drosophila basic helix-loop-helix transcription factor Atonal and is expressed specifically in retinal ganglion cells (Kay et al. Neuron. 2001 Jun. 30(3):725-36). In another embodiment, the promoter of the disease gene is replaced with the opsin promoter (Kennedy et al., (2001) J. Biol. Chem. 276(17): 14037-43) or rhodopsin promoter.

b. Ear Specific Expression.

Degeneration of a component of the auditory system affords a quantifiable screening system. Zebrafish display an acoustic startle, in which a sudden sound stimulus results in a reflex movement [Nicolson, 1998]. The intensity of a sound stimulus can be controlled in a graded fashion and the threshold for movement measured. Hundreds of fish can be tested at the same time. The invention additionally provides for monitoring the movement using a camera, e.g. a digital camera or video camera connected to a computer, to record positions of fish pre- and post-stimulus. This facilitates automation of the measuring process and screening of large numbers of fish. By combining this with a rescue strategy, it is possible to screen for the fish which retain the response, rather than losing it through degeneration of the auditory system [Bang, 2002]. Suitable ear-specific promoters include the scyba gene promoter [Long, 2000] and the NKX5-1 gene promoter [Adamska, 2000].

c. Pigmentation System.

Zebrafish adapt their skin colour to the ambient light intensity by varying their concentration and distribution of melanin. In a dark environment melanin granules become widely distributed within melanophores, star-shaped black pigment cells, thus causing the fish to look blacker. In contrast, in a light environment the melanosomes aggregate. The stimulus for this adaptation depends on a retino-hypothalamic projection, the hypothalamus then inducing pituitary secretion of either a hormone causing melanin aggregation or one causing melanin dispersal (Odenthal et al., 1996). The pigmentation level is readily apparent to the naked eye and can be quantitated readily with image analysis software such as NIH image. Thus, in the present invention assessment and comparison of pigmentation level may be used, again providing a high-throughput screening strategy to look for rescue of a defect generated by expressing a gene e.g. disease gene specifically in a component of the pathway controlling the pigmentation response. As part of this pathway involves neurons, neurological disease may be modelled specifically in a subset of neurons to give a graded, easily screenable phenotype which avoids deleterious effects.

Furthermore, the invention provides for an accurate, gradable and rapid screening method for the presence of disease activity and degree of disease activity. Thus in all cases, a continuous graded readout scale is achieved. This is highly desirable for identifying a therapeutic, as described earlier, and can be contrasted with phenotypic assays which measure an all or nothing response, such as the occurrence, or non-occurrence of seizures.

The fish, e.g. zebrafish, provided by the present invention are useful in screens for interactor, e.g. suppressor, genes that affect activity or effect of a second gene in the fish, such as a disease gene. According to a further aspect of the present invention there is provided the use of a fish, e.g. zebrafish, fugu, goldfish, medaka and giant rerio in such a screen.

As noted, model fish may be generated using chemical and/or physical means, in which case the invention also provides for screening for a gene that has an effect on the aspect of behaviour of physiology that is affected by the chemical or physical treatment.

Thus, the aspects of the invention involve genetic rescue of an induced phenotype.

Zebrafish are particularly amenable to genetic rescue experiments.

Mutagens such as ethylnitrosourea (ENU) may be used to generate mutated lines for rescue screening, in either the F1-3 (for dominant) or F3 (for recessive) generations. (It is only by the third generation that recessive mutations can be bred to homozygosity.) ENU introduces point mutations with high efficiency, so any phenotype is most likely to be recessive. Retroviral vectors may be used for mutagenesis, and although they are an order of magnitude less effective than ENU they offer the advantage of rapid cloning of a mutated gene (see e.g. Golling et al. (2002) Nat Genet 31, 135-40. Mariner/Tc family transposable elements have been successfully mobilised in the zebrafish genome and may be used as mutagenic agents (Raz et al. (1998) Curr Biol 8, 82-8. ENU remains the most efficient and easy method available at the moment, and so is preferred for now.

Rescue strains are then created and the underlying genes mapped. However, in the case of a dominant transgenic, gal4 driven disease or a HSP line, additional steps are preferably performed in accordance with aspects and embodiments of the present invention, as follows.

If the model is a dominant transgenic, then only 75% of the offspring from a cross of 2 adult transgenics will harbour the mutant gene. Thus in 25% of cases, one will not know whether rescue has occurred or whether no mutant gene has been inherited. Selective breeding strategies are therefore needed to generate a homozygous transgenic line. Heterozygous carriers are grown up and incrossed. Some of these progeny will be homozygous carriers. They can be identified as such through crossing to responder lines. They will be capable of driving expression of the responder gene in all cases in which the response element is inherited. The next step is then to mutate this line at random, such as with ENU, and to screen the offspring for deviation from the expected phenotype. All of the offspring of an outcross should be carriers. Any possible rescued fish are grown up then retested. Alternatively, an adult heterozygote is mutated at random and the offspring screened. Normal offspring, or those in which one suspects the phenotype may have been partially rescued, are genotyped from a sample of cells or tissue taken from the fish, e.g. via fin clipping or equivalent method. If the fish is a carrier, then it is grown up and the new line generated. The underlying rescue gene is then mapped.

If the model is under the UAS promoter, a related problem to that described above occurs. The solution in this case is to generate homozygous GAL4 driver lines, mutate these at random, cross these with the UAS lines, then screen for dominant rescue, as described in detail earlier.

The mapping of mutant genes is comparatively easy. The density of markers on the fish genetic map is already considerably greater than that of the mouse map, despite the relatively recent popularity of zebrafish. Consult the harvard website on zebrafish, findable using any available web browser using terms “zebrafish” AND “harvard”, currently (28 Nov. 2002) found at (http://zebrafish.mgh.harvard.edu/mapping/ssr_map_index.html), The Sanger Centre has begun to sequence the zebrafish genome with sequence currently (28 Nov. 2002) published at www.ensembl.org/Danio_rerio/. The site can be found using any web browser using terms “danio rerio” and “Sanger” or “ENSEMBL”. Around 70,000 ESTs have been identified and are being mapped on a radiation-hybrid map.

Another strategy for introducing effects, which may be random, on an aspect of behaviour or physiology in accordance with the present invention, is to down-regulate the function or activity of a gene, for instance employing a gene silencing or antisense technique, such as RNA interference or morpholinos. These can be either targeted against candidate genes, or generated against an array of genes as part of a systematic screen. It is relatively easy to inject RNA, DNA, chemicals, morpholinos or fluorescent markers into fish embryos, including zebrafish embryos, given their ex utero development.

A morpholino is a modified oligonucleotide containing A, C, G or T linked to a morpholine ring which protects against degradation and enhances stability. Antisense morpholinos bind to and inactivate RNAs and seem to work particularly well in zebrafish. Some disadvantages with this approach include the a priori need to know the gene sequence, the need to inject the chemical into the early embryo, potential toxic side effects and the relatively short duration of action. Additionally, they knock down the function of a gene, and thus do not offer the same repertoire of allele alterations as point mutations.

A further strategy for altering the function of a gene or protein as part of an in vivo screen, coupled to any of the various other components of the screening strategy disclosed herein, is to generate transgenic lines expressing protein aptamers, crossing these with the disease lines, or inducing disease by other means, then assaying for an altered disease state. Protein aptamers provide another route for drug discovery [Colas, 1996] but the ability to assay their effectiveness in vivo in accordance with the present invention markedly increasing their usefulness beyond in vitro screening methods.

In a further aspect, the present invention provides a method of screening for a suppressor gene that lessens activity or effect of a disease gene, the method comprising:
providing fish, e.g. zebrafish transgenic for a disease gene under regulatory control of a promoter, wherein expression of the disease gene within cells or tissue of the fish affects an aspect of behaviour or physiology of the fish, as model fish for screening;
subjecting said model fish to mutation to provide mutated fish;
comparing behaviour or physiology of mutated fish with behaviour or physiology of model fish in order to identify any mutated fish with altered behaviour or physiology compared with model fish;
identifying a genetic difference between model fish and any mutated fish with such altered behaviour or physiology, thereby to identify a suppressor gene that lessens activity or effect of the disease gene.

As noted, preferred embodiments of the present invention in its various aspects employ zebrafish.

A fish such as a zebrafish harbouring a disease-causing or phenotype-altering mutation manifested by altered behaviour or physiology as a result of expression of the disease gene may be identified on the basis that it does not display the behaviour or physiology or does not display it to the same degree because of the effect of a mutation in a second gene, this mutated second gene suppressing the activity of the disease gene.

Thus, in embodiments of the invention fish such as zebrafish transgenic for a disease gene expressed in the fish under control of a specific promoter (e.g. tissue-specific and/or inducible) may be maintained through several generations. This maintenance allows this strain to be entered into a secondary suppressor screen, through which a secondary mutation is introduced at random into the genome of this strain. The relevant aspect of behaviour or physiology of fish now harbouring both the disease causing mutation and the potentially suppressing secondary mutation may be assessed by means of a suitable assay. Fish in which relevant aspect of behaviour or physiology is better than the initial subjects of the mutation are candidates for harbouring a second mutation which is suppressing the effect of the primary disease causing mutation. This second mutated gene and its protein product are therapeutic targets for treatment of the disease caused by the primary mutation.

A mutant fish such as a mutant zebrafish transgenic for a disease gene under control of a particular promoter and containing a mutation within a suppressor gene that lessens activity or effect of the disease gene on an aspect of behaviour or physiology of the fish is itself useful in a further assay for a test substance able to modulate or affect, preferably potentiate or increase the suppression effect of the suppressor gene. Clearly, the same applies where a mutation in a gene is identified that enhances or increases activity of a second gene.

Of course, the person skilled in the art will design any appropriate control experiments with which to compare results obtained in test assays.

A number of strategies are available to the ordinary skilled person for introducing a secondary mutation into a primary mutant strain. These strategies may be applied in various situations in different embodiments of the present invention e.g. in mutation of gal4 driver lines or adult male heterozygous carriers). The mutagenesis procedure as described by van Eeden et al (Methods Cell Biol 1999 60) typically yields mutation rates of 0.9-3.3×10⁻³ per loci. A starting number of 100 healthy fertile males are considered necessary to obtain 20-30 fertile males after 6 treatments with 3 mM ENU.

Mutagenesis may be performed as follows:

Ethylnitrosourea (ENU) is dissolved in acetic acid to a final concentration of 10 mM, as determined by the optical density at 238 nm at pH6.0 (extinction coefficient=5830/M/cm), and then diluted to a working concentration of 3.0 mM in 10 mM sodium phosphate buffer, pH 6.6. Males which reliably produce fertilised offspring are placed in ENU solution for 1 hour. After the procedure the fish are washed in 2 changes of aquarium water for 1 hour each time, prior to return to the aquarium. The mutagenesis procedure is repeated up to 6 times at weekly intervals.

The frequency of mutations induced is proportional to the exact number of mutagenesis procedures performed. The number of procedures can thus be varied depending on the number of mutations desired per genome.

The actual mutagenesis procedure is best carried out in the dark to minimise the stress to the fish.

Initial progeny from the mutagenised fish are mosaic. The mutagenzed fish are therefore mated 3 times at weekly intervals following the final mutagenesis procedure. Progeny obtained after this will be non-mosaic, since any mutations will have arisen in spermatogonial stem cells.

Other useful mutagenesis agents include gamma- or X-ray-mediated mutagenesis, and retrovirus-mediated insertional mutagenesis.

An alternative method to overcome the problems of lethality and thus allow dominantly acting disease mutations to be entered into a secondary suppressor screen involves placing the mutated gene under the control of an inducible promoter, such as a heat shock promoter, the tetracycline inducible system (Clontech) or a hormonal inducible promoter.

The tetracycline inducible system (Clontech) relies on two different constructs. The tet-on plasmid expresses the tetracycline controlled transactivator, rtTA, consisting of a mutated tet-repressor protein, rTetR, coupled to the VP16 activation domain of the herpes simplex virus (AD). The rtTA protein binds to and activates the tetracycline response element (TRE) part of the second plasmid. The activated TRE acts with the CMV silent promoter to drive expression of the gene of interest. The key to the system is that the rtTA transactivator will only bind the TRE response element in the presence of tetracycline (or its analogue doxycycline). In the absence of tetracycline, binding does not take place, the response element is “off” and protein expression is minimal, such that even toxic proteins can be effectively switched on and off (Harkin et al Cell 1999 97:575-86; Lee et al PNAS 1988 95:11371-6).

The tetracycline inducible system has been used in mammals to provide regulated overexpression of interleukin 11 in the lungs of mice (Ray et al 1997 J Cline Inv 100: 2501-). There was no apparent toxicity to embryos treated with doxycycline in utero, or to pups or adults. In the absence of doxycycline levels of IL-11 were less than 50 pg/ml. Doxycycline induction raised levels to greater than 0.3 ng/ml. On removal of doxycycline from the drinking water IL-11 levels fell by >80% within 24 h. Alterations to the response element are possible, giving even tighter control.

A further modification involves the coupling of the TRE response element to a bi-directional promoter. This allows eGFP to be expressed whenever the target gene is also expressed. This provides an easy visual marker of gene carriage and activation.

Thus in the scenario of a secondary suppressor screen, a mutated gene is introduced into the fish, e.g. zebrafish, under the control of an inducible system (e.g. tetracycline inducible). The gene is switched on by addition of the inducer (e.g. tetracycline) and those fish expressing the gene selected for further study. The gene is then switched off, allowing the fish to be reared through subsequent generations and thus allowing additional mutations to be introduced as part of a secondary suppressor screen.

Accordingly, in a further aspect, the invention provides a method of screening for a suppressor gene that lessens activity or effect of a disease gene, the method comprising:
providing fish such as zebrafish as model fish for screening, which model fish are transgenic for a gene construct wherein a coding sequence a for disease gene that is lethal in fish or renders fish non-viable or non-reproductive is provided under control of an inducible promoter and the model fish are generated and reared under conditions in which the inducible promoter is not induced and/or is repressed;
subjecting the model fish to mutation to provide mutated fish and inducing and/or de-repressing the inducible promoter to cause expression of the disease gene in the model fish; determining viability and/or reproductivity of mutated fish in which the disease gene is expressed in comparison with model fish in which the disease gene is expressed; identifying a genetic difference between model fish and any mutated fish with altered viability and/or reproductivity, thereby to identify a suppressor gene that lessens activity or effect of the disease gene.

Such a method may be employed in combination with for example the gal4/UAS system for added spatial control, or with a specific combination of known or unknown chemicals, as disclosed herein.

The suppressed strain of fish will have at least two mutations: the disease-causing/phenotype-inducing mutation resulting from introduction of the disease gene, and the disease-/phenotype-suppressing or repressing mutation.

It should be noted also that in place of identification of a suppressor mutation and gene, the present invention in any of its aspects and embodiments may be used to identify a mutation and gene that enhances or increases the severity of the primary phenotype. This can be used to identify additional genes involved in a particular disease pathway. The term “interactor gene” may be used to denote a gene that affects activity or effect of a primary gene.

Similarly, the present invention may be used to identify a test substance that affects activity or effect of a gene in a fish or a treatment of a fish, e.g. where the gene is a transgene expressed in a tissue or under certain conditions in the fish and the transgene or the treatment has an effect on an aspect of behaviour or physiology of the fish.

A screening or assay method according to an aspect or embodiment of the present invention may comprise identifying a suppressor gene that lessens activity or effect of a disease gene.

A screening or assay method according to an aspect or embodiment of the present invention may comprise identifying a gene that enhances or increase activity or effect of a second gene, or a test substance that affects activity or effect of the second gene, whether lessening or decreasing, or enhancing or increasing such activity or effect.

Gene identification may be facilitated by taking advantage of the easy of transplanting cells from one fish embryo to another, e.g. zebrafish, given the ex utero development. By labeling donor cells, for example by injecting a fluorescent marker into the early embryo, it is possible to trace the fate of the donor cells. This may be used to identify whether a gene acts in a cell-autonomous manner to be asked. For example, if a mutant cell no longer degenerates when surrounded by wild-type cells, this suggests the gene acts in a non-cell autonomous manner, and thus may code for a secreted or cell surface protein. In one example, eye bud or lens transplants may be used to determine whether a defect is intrinsic to the retina or to the lens, and to disentangle lens-retinal signaling processes [Link, 2001].

Following identification of a gene which affects activity or effect of a second gene, e.g. a suppressor gene, the gene (including a homologue in another species, e.g. human) or encoded gene product may be cloned or otherwise provided in an isolated or purified form, and may be provided in a composition comprising at least one additional component.

Often there will already be enough confidence in similarities in biological pathways to move straight to human or another mammal. However, certain steps may help.

Where there is a mutated gene leading to rescue, the human homologue of that gene may be introduced into the rescue line in both wild-type and mutated form. If the human gene has equivalent action in its mutated form, then rescue will be seen when it is injected in the mutated form, but may be lost when injected in the wild-type form, depending on the mechanism of action of the mutated gene.

Where drugs are already known to act against the rescuing gene or its encoded protein, these can be screened directly. As this is easy to do because of the attributes engineered into the system, as disclosed herein, this is quicker to do than embarking on an exploration of the equivalence of biological pathways.

Where only possible drugs are known that act against related proteins to the rescuing encoded protein, then these can all be screened. Again, the scalability of the system described above makes this a cost-effective way to proceed.

Where the rescuing protein proves to be a poor target, or where a rescuing protein remains elusive, gene and protein microarrays and gene and protein profiling techniques may be used to identify potential targets. These approaches can generate many false leads and conventionally require much work to identify real lead candidates. However, using the present invention, the effort required to screen candidate drugs or chemicals against dozens or hundreds of possible targets is less than that required to further validate these individual targets.

As noted, the gene, e.g. suppressor gene, (including a homologue in another species, e.g. human) or a gene product encoded by the gene, e.g. suppressor gene, may be used in a screening system for assaying ability of a test substance to affect activity of the gene or the gene product encoded by the gene.

A test substance that affects activity of a gene, e.g. a suppressor gene, or the gene product encoded by the gene may be provided in a composition comprising at least one additional component.

Following identification of a suppressor gene for a disease gene of interest, or other gene that affects activity or effect of a second gene, the suppressor or other gene and/or an encoded gene product may be employed as a target for identification of potential therapeutics or as a therapeutic in its own right. Also, the nature of the suppressing or other effect may be investigated further.

The suppressor or other gene that affects activity or effect of a second gene may be a novel gene or may be a known gene not previously known to have a function of affecting or suppressing activity or effect of the relevant disease gene. The gene may be one already known or suspected to have function in affecting or suppressing activity, in which case the results from the fish assay add weight to the available evidence. In particular, the fact that the suppression or other effect occurs in vivo increases the confidence for using the gene, or encoded gene product or fragment thereof, or a component in the pathway of action of the gene or gene product, as a drug target. For further investigation and use, a homologue from another species may be used, where available e.g. via use of cloning or screening technology.

The responsible mutation, e.g. suppressive mutation, may be identified by using mapping techniques available in the art, (e.g. see Detrich H. W., Zon L. I. & Westerfield M. (1998) The Zebrafish: Genetics and Genomics. Methods in Cell Biology. Volume 60, pg 182-192).

Thus, to identify the position of the relevant mutation, e.g. a suppressive mutation, the mutant locus is mapped relative to the position of a marker, the position of which is known. DNA markers include short sequences of DNA, cloned genes or other mutations. The current best method in zebrafish involves simple sequence length polymorphisms (SSLPs) as they cover the entire genome at high density. It is therefore possible to map to within 0.5 cM, from which either a chromosomal walk may be initiated, further mapping may be undertaken using single strand conformational polymorphisms, or candidate genes selected directly.

Mapping Using SSLP

These markers consist of 2 primers flanking a dinucleotide (CA) repeat. These are extremely variable in length & polymorphic between zebrafish strains. The SSLP mapping involves the following steps:

Raising a map cross, identifying mutant carriers, fixing mutant & sibling progeny separately

Isolating genomic DNA from both mutants & siblings

Genome scanning using pooled DNA from both mutants & siblings to determine linkage group

Verifying potential linkages with single embryo DNA

Searching for closely linked markers

Positioning the mutation on the genetic map by determining the number of recombinations between marker & mutation.

Isolation of Genomic DNA

To extract DNA from single embryos, embryos fixed in 100% methanol are poured into a petri dish. More methanol is added to the dish to ensure the embryos remain covered. Embryos are then pipetted into a 96 well plate: a single embryo per well. A pipette is then used to remove as much methanol as possible from around the embryos. The remaining methanol is then evaporated off on a PCR block set at 70° C. for 15 minutes. 25 ul of a mix of 250 ul proteinase K (17 mg/ml, Merck) & 2.25 ml 1×TE, is added to each well. The PCR plate is then covered with Hybaid film & heated in a PCR machine for 240 minutes at 55° C., followed by a 10 minute 75° C. incubation to inactivate the proteinase K. The plates can be kept at −20° C. until needed.

Genome Scanning

Pooled DNA is prepared by taking 10 ul from each of 48 single samples, and then diluted to a final concentration of 50 ng/ul. Primers for markers are arranged on a master primer 96 well plate in such a way that the mutant & sibling sample analysed with the same marker will subsequently run adjacent to each other on an agarose gel. The markers selected for the PCR plates are those known to show useful polymorphisms & which evenly span the entire genome.

PCR reactions are then set up in 96 well format. Each well contains 14.28 ul PCR mix, 0.16 ul each of 20 uM forward & reverse primer, 0.4 ul of 5 U/ul Taq polymerase & 5.0 ul of template DNA. PCR is performed with initial denaturing at 94° C. for 3 minutes, followed by 35 cycles of denaturing at 94° C. for 30 seconds, annealing at 60° C. for 30 seconds & primary extension at 72° C. for 1 minute. The reaction is completed by a final 5 minute extension at 72° C.

PCR Mix

0.2 mM dATP

0.2 mM dCTP

0.2 mM dGTP

0.2 mM dTTP

in PCR buffer

PCR Buffer (10×)

100 mM Tris-HCl, pH 8.3

500 mM KCl

15 mM MgCl₂

0.1% (w/v) gelatin

Single-Embryo PCR

PCR reactions are set up and performed as above, except that single embryo DNA is used as the template.

The PCR products are assessed for polymorphisms by running out on a 2% agarose gel at 200V for 80 minutes in 1×TBE.

Mapping Using SSCP

This uses single strand DNA. Each strand assumes its thermodynamically preferred conformation. Single nucleotide substitutions may alter the conformation sufficiently for a difference in migration pattern to be detected on a non-denaturing gel. This allows non-SSLP markers tightly linked to the mutation to be analysed.

The protocol used to amplify a marker is as for SSLP mapping. To precipitate the PCR products, 2 volumes of pre-cooled 100% ethanol and 0.1 volume of 3M Na-acetate are added to the PCR product, vortexed well, incubated for at least 20 minutes at −20° C. & centrifuged in a cooled centrifuge at 13000 rpm for 25 minutes. The supernatant is discarded, the DNA pellet air-dried & resuspended in 8 ul of ddH₂O. To 5.4 ul of PCR product, 0.6 ul of denaturing solution & 2.4 ul of loading buffer are added & briefly mixed, prior to incubation at 85° C. for 10 minutes. The sample is then quickly chilled on ice. 6-8 ul of each sample is then loaded onto a native precast acrylamide gel (CleanGel SSCP, ETC Elektrophorese-Technik) & run at 200V & 15° C. following the manufacturer's instructions.

Denaturing Solution

10 mM EDTA

500 mM NaOH

Loading Buffer

2% bromophenol blue

2% xylenecyanol

in formamide

The gels are then stained using a silver staining kit (PlusOne DNA Silver Staining Kit, Pharmacia), as per the manufacturer's instructions.

By these methods the mutation is mapped close enough to select a candidate gene. This gene is then sequenced in both mutant wild-type fish to identify mutations.

If the suppressor or other gene that affects activity or effect of a second gene encodes a protein, it may be that that protein interacts with or binds the second gene, e.g. disease gene, or gene product. Thus, for example, a novel protein-protein binding pair may be identified, immediately presenting the possibility of modulating or affecting such binding as a target for identifying candidate therapeutics.

Where interaction or binding between gene products is to be investigated further or employed in assay methods for identifying further substances able to affect the binding or interaction, suitable approaches are available in the art, for instance techniques involving radioimmunoassay, co-immunoprecipitation, scintillation proximity assay, ELISA methods, and two-hybrid assays (see e.g. Fields and Song, 1989, Nature 340; 245-246), for instance using the two binding domains of the GAL4 transcription factor or the LexA/VP60 system.

Further mutation in the suppressor or other gene may be used to identify variants with enhanced or otherwise altered suppressor function.

Thus, the suppressor gene or other gene, or encoded gene product, in wild-type or a mutated form (which may be a mutated form as identified in the original screen or a further mutated form) may be used in a therapeutic composition.

In various further aspects, the present invention thus provides a pharmaceutical composition, medicament, drug or other composition comprising a suppressor gene or other gene or gene product or substance found to affect the disease gene of interest or suppression of the disease gene of interest, the use of such a material in a method of medical treatment, a method comprising administration of such a material to a patient, e.g. for treatment (which may include preventative treatment) of a medical condition, use of such a material in the manufacture of a composition, medicament or drug for administration for such a purpose, e.g. for treatment of a proliferative disorder, and a method of making a pharmaceutical composition comprising admixing such a material with a pharmaceutically acceptable excipient, vehicle or carrier, and optionally other ingredients.

One or more small molecules may be preferred therapeutics identified or obtained by means of the present invention. However, the invention may be used to identify appropriate targets for antibody mediated therapy, therapy mediated through gene targeting or protein targeting, or any of a variety of gene silencing techniques, including RNAi, antisense and morpholinos.

Whatever the material used in a method of medical treatment of the present invention, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Examples of techniques and protocols mentioned above can be found in Remington's Pharmaceutical Sciences, 16th edition, Osol, A. (ed), 1980.

Vectors such as viral vectors have been used in the prior art to introduce nucleic acid into a wide variety of different target cells. Typically the vectors are exposed to the target cells so that transfection can take place in a sufficient proportion of the cells to provide a useful therapeutic or prophylactic effect from the expression of the desired peptide. The transfected nucleic acid may be permanently incorporated into the genome of each of the targeted cells, providing long lasting effect, or alternatively the treatment may have to be repeated periodically.

A variety of vectors, both viral vectors and plasmid vectors, are known in the art, see U.S. Pat. No. 5,252,479 and WO 93/07282. In particular, a number of viruses have been used as gene transfer vectors, including papovaviruses, such as SV40, vaccinia virus, herpesviruses, including HSV and EBV, and retroviruses. Many gene therapy protocols in the prior art have used disabled murine retroviruses.

As an alternative to the use of viral vectors in gene therapy other known methods of introducing nucleic acid into cells includes mechanical techniques such as microinjection, transfer mediated by liposomes and receptor-mediated DNA transfer, also administration of naked DNA or RNA, by simple administration, e.g. injection, of nucleic acid such as a plasmid, for instance to muscle.

The following sets out in detail certain embodiments of aspects of the present invention.

A disease model may be induced by genetic means.

In preferred embodiments the disease gene is placed under the control of a UAS promoter.

1. Clone the desired promoter (e.g. rhodopsin) upstream of a gal4 coding sequence to produce an expression construct or transgene.

2. Inject the construct into wild-type zebrafish embryos.

3. Grow up injected embryos.

4. Genotype growing embryos, for example using PCR on DNA extracted from a fin clip of juvenile zebrafish, using primers which amplify a component of the injected construct. Alternatively or in addition, founders can be identified through crossing offspring with a UAS responder line and assessing for gene expressing in the progeny.

5. Adult gal4 driver line males are then exposed to mutagenesis, e.g. with ENU.

6. The coding sequence of a disease causing gene is cloned downstream of a UAS promoter.

7. Transgenic zebrafish expressing this construct are then generated.

8. The mutagenised gal4 driver males are then crossed with responder females. 25% of the offspring will express the mutant gene and thus should express the mutant phenotype. They are screened both for the expression of the mutant gene (e.g. through detection of GFP tagging, through genotyping or through partial disease expression), as well as for phenotypic expression. If the phenotype is less than expected, the mutation in the gal4 driver line may be rescuing the underlying phenotype.

9. The “rescued” fish is grown up to adulthood and outcrossed to a wild-type strain. 12.5% of the offspring will also be genotypically diseased fish, but phenotypically rescued.

10. These are identified as above and are subsequently crossed to a wild-type strain, polymorphic with respect to the original founder strain.

11. The progency of the subsequent generation is sorted into genotypically diseased fish and genotypically wild-type fish and used for mapping of the underlying rescue gene, by conventional methods.

12. The protein encoded by the human homologue of this gene is used as a target for drug discovery—chemicals which bind to this target and have an equivalent effect on function as the mutation, will be expected to have a similar beneficial effect on phenotype.

In another embodiment of the present invention, a disease-causing gene is placed under the control of an inducible promoter, such as a heat shock promoter.

1. Clone the coding sequence of the disease gene, e.g. huntingtin, downstream of heat shock promoter.

2. Inject construct into embryos.

3. Identify founder embryos, e.g. through PCR on DNA extracted from fin clips or through detection of a marker gene expressed along with the disease-causing gene.

4. Out-cross adult mosaic founders to generate transgenic lines.

5. Mutagenise adult male carriers of subsequent generation.

6. Induce the expression of the disease causing gene in the offspring of these founders through the elevation of the temperature of the water.

7. Assay for the disease phenotype.

8. Those fish which do not have the expected disease phenotype severity may have been rescued.

9. Grow these fish up and out cross. 25% of the offspring will also be disease resistant.

10. These are subsequently used for mapping of the underlying gene.

In another embodiment, a disease phenotype is introduced into fish through random mutagenesis. The following sequential steps may be performed:

1. Mutagenesis e.g. with ENU of adult male wild-types.

2. Screening of F1, F2 or ideally F3 progeny for phenotype of interest.

3. Characterisation of phenotype to confirm disease relevance.

4. Ideally mapping of underlying gene.

5. Secondary mutagenesis of adult male heterozygotes.

6. Crossing of these mutagenised heterozygotes with female heterozygotes.

7. Screening of the progeny from this cross for the initial phenotype.

8. If the phenotype is deviates from that in the original model fish, then some rescue has taken place.

9. Given that if there is complete rescue, one would not identify it, an alternative method involves genotyping of each progeny, e.g. via analysis of a sample obtained from a fish such as through fin clippings. If a fish is genotypically mutant, but phenotypically wild-type, it has been rescued.

10. The rescued animal is then grown up and outcrossed to generate new lines. These are then used to map the underlying gene and develop a therapeutic, as described above.

In a further embodiment, the present invention provides a variation of this methodology wherein the identification of a diseased strain is performed as follows:

1. Random mutagenesis, such as with ENU.

2. Extraction of DNA from the F1 progency.

3. High-throughput genotyping to identify a fish with a mutation in a desired gene.

4. Isolation of a strain derived from this fish.

5. Phenotype analysis to see whether the mutation is pathogenic.

6. This strain is then used as above.

In a still further embodiment, the invention provides another variation of this methodology which involves creation of a diseased strain through an antisense or other gene silencing technique, as follows:

1. Random mutagenesis of wild-types, e.g. with ENU.

3. Inject F1 embryos with a gene silencing agent, e.g. morpholino, known to induce a desired phenotype, or RNAi or antisense RNA.

4. Screen for deviation from initial phenotype.

5. If phenotype deviates, grow up embryo and generate line.

6. Rescreen next generation and sort into mutant and sibs, map and use as above.

In a still further embodiment, F2 or F3 embryos may be injected and rescue assessed based on partial deviation from initial phenotype, or population effects. Then, as above.

In various aspects of the present invention, embodiments employ alternative methods of generating the biological state, for example including any of the following non-genetic models:

1. Wild-type fish are mutagenised, e.g. by ENU, and suitable progeny screened for deviation of the expected phenotype. This will typically be F3 progeny. The biological state may be induced by one of the following methods:

Application of a Chemical to Induce a Disease Phenotype.

For example, day 5 wild-type zebrafish embryos are exposed to streptozotocin to induce necrosis of the pancreatic beta cells, in order to induce a state resembling type 1 diabetes.

Application of a Physical Agent to Induce a Disease Phenotype.

For example, zebrafish, e.g. at day 21 are exposed to white light to induce degeneration of photoreceptors and a state resembling retinal degeneration (Vihtelic & Hyde, J Neurobiol, 2000, PMID: 10942883).

Elucidation of a Behavioural Response.

For example, fish are rendered addicted to a substance through exposure to that substance for several days. For example, nicotine is added to the fish water from days 3-5. The water is then replaced with fresh water. On day 7 the embryos display addictive behaviour through the preferential swimming towards a source of nicotine. The extent of their addiction can be assessed by exposing the embryos to a second signal, tending to move the fish away from the source. This could involve the passage of stripes away from the source of nicotine. This evokes an optomotor response, with the fish swimming with the stripes. It may involve a food source or a temperature gradient in the water. There are thus two conflicting pulls for the fish. If the fish is very addicted, it will swim to the nicotine. Otherwise, if the optomotor stimulus is sufficiently strong, it will swim with the stripes. Any opposing stimuli could be used.

Those fish which deviate from the expected or initial phenotype may have been rescued. The parents of the progeny may be outcrossed to a wild-type strain of the same background, and the subsequent generation retested, to ensure the rescue breeds true.

The parents may also be outcrossed to a wild-type polymorphic strain.

The progeny of this outcross may then be sorted into rescued “mutants” and sibs and used for mapping the underlying gene through conventional methods. The gene may then used for derivation of a therapeutic, as described above.

Alternatively, instead of or as well as an attempt to rescue the phenotype through the induction of genetic mutations, rescue may be achieved through application of a test substance, e.g. one or more chemicals. In this situation, all of the above methods will not require the mutation step for rescue (but will still require the mutation step if this is part of the procedure for induction of the disease state). The procedure may then be as follows, in accordance with embodiments of the present invention:

1. A test substance is added to the fish either prior to the appearance of the disease state, at the time of induction of the disease state, or after the induction of the disease state. The first two situations are more likely to identify a prophylactic chemical, the latter a drug which reverts the disease state back to normal. The test substance may be a chemical and may be a random chemical administered in a high-throughput fashion to fish in 96 well plate format, or a selected chemical administered to a clutch of fish in a Petri dish.

2. The fish is then screened for deviation from the initial disease state.

The following additional steps are highly desirable in screening, and their use is provided by the present invention in preferred embodiments:

Rather than add a single chemical, a combination of chemicals is added. For instance, a known therapeutic agent may be administered to all fish at a dose at which a further beneficial effect could still be detected. A random chemical library is then added to fish and an incremental effect screened for.

To give an even more specific example, epilepsy may be induced in fish (e.g. 7 day old) using pentylene tetrazole, for example at a concentration of 15 mM. A known anti-epilepsy drug, e.g. sodium valproate or diazepam, may be added to all fish. Test substances, for instance from a random or combinatorial chemical library, may be added to individual wells. The screen is then for deviation from the expected phenotype (a certain degree of amelioration of the seizure symptoms) obtained by just adding the known anticonvulsant after 16 hours.

When augmentation is seen, one knows that the test substance has a beneficial action in vivo with a known anticonvulsant, potentially synergistic. This is highly desirable, given that clinical trials involving add-on drugs are much easier to conduct.

In a further embodiment of the invention, a panel of test substances, e.g. known or potential therapeutic agents, are screened in various combinations.

This may be used for example to determine an advantageous or optimum combination of a number of different therapeutics (e.g. anticonvulsants), by adding every possible combination of the substances in an array fashion to the epileptic fish.

Continuing with epilepsy purely as an illustrative example, a typical protocol may be as follows:

1. Induce epilepsy with pentylene tetrazole at a concentraion of 15 mM in 7 day old fish.

2. Administer drug 1 to row 1, drug 2 to row 2, etc.

3. Administer drug 1 to column 1, drug 2 to column 2 etc.

4. Compare the anti-epileptic effect of all wells with that of the drugs when given alone.

A further embodiment of the above allows for detection of augmentation of a particular drug through a particular mutation, as follows:

1. Induce genetic mutation through any of the above.

2. Induce disease state through any of the above.

3. Administer test chemical.

4. Assess whether the combination of the mutation plus chemical is greater than either alone.

5. The mutated gene is then used as a beneficial target, as described above.

A further embodiment of the invention allows identification of genetic factors which help determine the appropriateness of a particular therapeutic agent for a given patient. If the mutation augments the effect of the drug, that mutation is searched for in human homologues. Patients with this mutation should be preferentially prescribed the drug. If the mutation leads to a deleterious effect or lack of effect, then patients should avoid this drug.

A further embodiment of the invention allows identification of genetic or chemical factors which help prevent the side effects of an otherwise toxic drug. The following is an illustrative embodiment with reference to multiple sclerosis, but may be applied in other contexts for other diseases:

1. Mitoxantrone has a beneficial effect on multiple sclerosis (MS) patients, but causes cardiotoxicity.

2. An MS model of zebrafish is created which responds to treatment with mitoxantrone, but with the added complication of cardiac pathology.

3. The treated fish are co-treated with a panel of chemicals, (or alternatively are mutagenised as a route to a drug target).

4. Those fish which no longer show the cardiac effects, but still show the beneficial effects are selected. The chemical is then used as a co-agent in patients to allow the safer administration of the mitoxantrone, (or alternatively the mutagenised gene is mapped and used to develop the co-agent).

A further embodiment of the present invention involves attempting to modify the initial phenotype through a protein aptamer, rather than through a genetic mutation of chemical means. For example, a method may be performed in accordance with the following:

1. A construct coding for the desired aptamer (or random constructs for random aptamers) is injected into embryos to generate lines expressing the aptamer.

2. These lines are then crossed to the disease-expressing lines, or alternatively the disease state is induced in these lines.

3. The lines are then tested for deviation from the expected or intial phenotype.

4. If deviation occurs, the aptamer has in vivo proof of action and is used to derive a therapeutic agent.

Having identified fish with a mutation that confers rescue on a disease phenotype, the following steps may be performed:

1. The human homologue of the zebrafish rescue gene is cloned.

2. The same type of mutation is introduced into the human homologue

3. The wild-type and mutated constructs are injected into the embryos.

4. The disease state is induced and assessed.

5. If the wild-type gene prevents the rescue, but the mutant gene retains it, this provides further evidence that the mutation is beneficial. However, a negative result does not necessarily rule out benefit.

6. The protein encoded by the human homologue is used for direct drug screens in vitro or directed in vivo screening.

Zebrafish are DMSO Tolerant

The inventors have discovered that zebrafish are DMSO tolerant. This is important as many drugs are dissolved in DMSO (dimethyl sulphoxide). The inventors have established that zebrafish can tolerate 1% DMSO. Thus, a candidate drug or other test substance may be dissolved in DMSO and administered to zebrafish by adding to the fish water to give a final concentration of DMSO of at least upto 1%. This is employed in various preferred aspects and embodiments of the present invention.

Zebrafish of tup background were tested in batches of 10. DMSO was added to fish water on day 3 and fish examined daily up to day 9. At 1% DMSO (10 ul/ml), no abnormality was detected, as assessed by detailed examination under a dissecting microscope to 5× magnification. Motoric behaviour was also normal.

Generation of a Disease Model

The 1.2 KB rhodopsin promoter described by [Kennedy, 2001] was cloned into pEGFP-1 (Clontech vector accession number U55761) at EcoR1/Sal1 (rhodopsin accession number: AF331797). The Crx promoter (accession number: AF301006) was supplied by Furukawa [Furukawa, 2002]. The 2 kB promoter was cloned at a single Sma1 site. The huntingtin constructs consisted of exon 1 with 21 (21Q) and 72 CAG repeats (74Q), cloned into pEGFP-C1 at BglII/EcoR1 sites, as described by [Wyttenbach, 2000]. The EGFP and huntingtin constructs were excised from pEGFP-C1 using Age1 and Mfe1, then cloned downstream of the rhodopsin promoter at same sites (i.e. GFP from pEGFP1 removed). Xho1 and BfrI were then used to excise a fragment for injection. This was gel purified and 1-3 nl of a 100 ug/ul concentration injected into 1 to 8 cell stage embryos. Eye specific expression was assessed through microscopy of MS222 anaesthetised embryos.

The Gal4 and UAS constructs were as described in (Brand, 1993]. The constructs were subcloned into pBluescript (Stratagene pBluescriptII KS) at a single HindIII site or at Pst1/Cla1 site. The Crx promoter, cut out of the original vector with SmaI, was ligated in at a single Sma1 site, then sequenced to determine direction. Additionally the rhodopsin promoter was cut out of original vector with SacI/SacII, then ligated in at the same sites. UAS was cloned into pBluescript at a single Pst1 site. The huntingtin constructs were cut out of pEGFP-C1 with Eco47III (blunt) and SalI, then cloned into pBS-UAS at EcoRV (blunt) and SalI.

Use of a Temperature Gradient

Experimentally, a temperature gradient was created and certain tests run:

A water temperature gradient was generated. It was observed that fish displayed an aversion to hotter temperatures, preferring temperatures less than 29.5° C. and specifically avoiding temperatures less than 32° C.

It was also observed that fish prefer better illuminated areas and swim away from dark areas.

It was also observed that fish will swim with moving stripes.

We observed that when fish were exposed to both a temperature gradient (from 24 to 40° C.) and either a variation in illumination of moving stripes, that the latter stimuli could overcome the former stimuli, with the result that the fish moved to temperatures beyond 32° C.

Nicotine Addiction Experiment

Fish were reared in nicotine from 2 dpf (immediately after hatching) to 7 d.p.f. at concentrations (in the fish water) of 0.5, 2.5, 5, 25, 50 ug/ml (5 fish per test group). The nicotine medium was then removed and replaced with E3M (embryo medium) at 7 d.p.f. They were then challenged at 8 d.p.f. with 2.5 ug/ml nicotine in 3% methyl cellulose.

Controls, as well as those embryos previously bathed in 0.5 and 2.5 ug/ml showed no response/preference to nicotine. Toxicity was seen at doses of 25 and 50 ug/ml. Those previously bathed at 5 ug/ml responded as shown in the figure.

2 fish were attracted to the nicotine impregnated methyl cellulose by 4 mins, 3 fish by 5 mins and 4 fish by 15 mins, preferentially positioning themselves there. In contrast, fish exposed to non-impregnated methyl cellulose, as well as fish not previously exposed to nicotine but exposed to nicotine-impregnated methyl cellulose, did not position themselves preferentially.

Details of the toxicity dose/response experiment are given below:

Dose/tox Data at 3 d.p.f.

0—all alive, escape response when prodded

0.5—all alive, escape response as above

2.5—all alive, escape response as above

5.0—all alive, escape response as above

25—all alive at 3 d, twitch but no escape response when prodded

50—all alive at 3 d, twitch but no escape response when prodded

Dose/tox Data at 4 d.p.f.

0— all alive, escape response when prodded

0.5—all alive, escape response as above

2.5—all alive, escape response as above

5.0—all alive, escape response as above

25—4 dead, 1 alive, twitch but no escape response when prodded

50—all, dead

All documents mentioned anywhere in this specification are incorporated by reference.

### REFERENCES

Adamska, et al. (2000) Mech Dev 97, 161-5.
Bang et al. (2002) J Neurosci Methods 118, 177-87.
Brand et al. (1994) Methods Cell Biol 44, 635-54.
Brand and Perrimon (1993) Development 118, 401-15.
Calissendorff (1976) Acta Ophthalmol (Copenh) 54, 109-17.
Colas et al. (1996) Nature 380, 548-50.
D'Avino and Thummel (1999) Methods Enzymol 306, 129-42.
Driever et al. (1996) Development 123, 37-46.
Duffy (2002) Genesis 34, 1-15.
Eells (2000) Neurotoxicology 21, 321-30.
Furukawa et al. (2002) J Neurosci 22, 1640-7.
Ghalie et al. (2002) Neurology 59, 909-13.
Goldman et al. (2001) Transgenic Res 10, 21-33.
Gong et al. (2002) Dev Dyn 223, 204-15.
Haffter et al. (1996) Development 123, 1-36.
Hassan and Janjua (2001) J Ayub Med Coll Abbottabad 13, 26-30.
Heng et al. (1999) Invest Ophthalmol Vis Sci 40, 190-6.
Higashijima et al. (2000) J Neurosci 20, 206-18.
Higashijima et al. (1997) Dev Biol 192, 289-99.
Huang et al. (2001) Mol Cell Endocrinol 177, 117-24.
Ju et al. (1999) Dev Genet 25, 158-67.
Kelley et al. (1995) Development 121, 3777-85.
Kennedy et al. (2001) J Biol Chem 276, 14037-43.
Lawson and Weinstein (2002) Dev Biol 248, 307-18.
Link et al. (2001) Dev Biol 236, 436-53.
Long et al. (1997) Development 124, 4105-11.
Long et al. (2000) Mech Dev 97, 183-6.
Meng et al. (1997) Proc Natl Acad Sci USA 94, 6267-72.
Meng et al. (1999) Blood 93, 500-8.
Milewski et al. (1998) Endocrinology 139, 1440-9.
Nicolson et al. (1998) Neuron 20, 271-83.
Odenthal et al. (1996) Development 123, 391-8.
Perkins et al. (2002) Vis Neurosci 19, 257-64.
Peterson et al. (2000) Proc Natl Acad Sci USA 97, 12965-9.
Posner et al. (1999) Biochim Biophys Acta 1447, 271-7.
Saszik et al. (1999) Vis Neurosci 16, 881-8.
Scheer et al. (2002) Mech Dev 112, 9-14.
Solnica-Krezel et al. (1994) Genetics 136, 1401-20.
Taurog (1976) Endocrinology 98, 1031-46.
Tokuoka et al. (2002) J Neurosci 22, 10324-32.
van de Wyngaert et al. (2001) Acta Neurol Belg 101, 210-6.
Vihtelic and Hyde (2000) J Neurobiol 44, 289-307.
Wyttenbach et al. (2000) Proc Natl Acad Sci USA 97, 2898-903.
Yeh and Hsu (2000) (Danio rerio). Biosci Biotechnol Biochem 64, 592-5.

1. A method of screening for a substance or first gene that affects activity or effect of a second gene, or activity or effect of a treatment, or activity or effect of a physiological function or behaviour, on a fish, the method comprising:
providing, as model fish for screening, (i) fish transgenic for the second gene, wherein the second gene is under regulatory control of a specific promoter and expression of the second gene within the fish affects an aspect of behaviour or physiology of the fish, or (ii) fish subject to said treatment, wherein the treatment affects an aspect of behaviour or physiology of the fish;
mutating said model fish to provide a genetically mutated fish or treating said model fish with a test substance to provide treated fish;
comparing an aspect of behaviour or physiology of mutated fish or treated fish with that aspect of behaviour or physiology of model fish in order to identify any mutated fish or treated fish with altered behaviour or physiology compared with model fish;
thereby to identify a test substance that affects activity or effect of the second gene or activity or effect of said treatment, or, by identifying a genetic difference between model fish and mutated fish with such altered behaviour or physiology to identify a first gene that affects activity or effect of the second gene or activity or effect of said treatment;
wherein the mutated fish or treated fish, and model fish, are subject to two opposing stimuli, and the aspect of behaviour or physiology of the mutated fish or treated fish subject to the two opposing stimuli is compared with the aspect of behaviour or physiology of model fish subject to the two opposing stimuli.

2. A method according to claim 1 wherein either or both of the two opposing stimuli are selected from any combination of the members of the group consisting of: a visual stimulus, light stimulation, optomotor stimuli, an audible startle stimulus, discrete changes in temperature, a temperature gradient, food, at least one aversive substance, at least one attractive or additive substance, physical aversion and a threatening shape.

3. A method according to claim 1 wherein fish are habituated to an addictive substance, and one of the two opposing stimuli is provided by a source of the addictive substance to the fish.

4. A method according to claim 3 wherein the addictive substance is nicotine.

5. A method according to claim 1 wherein one of the two opposing stimuli is provided by a temperature gradient in water inhabited by the fish.

6. A method according to claim 5 wherein the temperature gradient is from 24° C. to 40° C.

7. A method according to claim 1 wherein one of the two opposing stimuli is provided by an inducer of the optomotor response.

8. A method according to claim 7 wherein the inducer is moving stripes.

9. A method according to claim 1 comprising treating fish with at least two substances, at least one of which is a test substance, and comparing the effect of the at least two substances in combination, whether simultaneously or sequentially applied, on the aspect of behaviour or physiology of treated fish with the effect of at least one of the two or more substances when applied individually or alone.

10. A method according to claim 9 wherein treated fish are transgenic and/or mutated.

11. A method according to claim 1 comprising providing fish transgenic for the second gene, wherein the second gene is under regulatory control of at least one of an inducible promoter and a and tissue-specific promoter.

12. A method according to claim 11 wherein the promoter directs eye-specific expression.

13. A method according to claim 11 wherein the promoter directs ear-specific expression.

14. A method according to claim 1 comprising determining auditory function of the fish.

15. A method according to claim 1 comprising determining visual function of the fish.

16. A method according to claim 1 comprising determination of pigmentation of the fish.

17. A method according to claim 1 further comprising formulating a substance that affects activity or effect of said second gene, or activity or effect of said treatment, into a composition comprising at least one additional component.

18. A method according to claim 1 comprising identifying said first gene.

19. A method according to claim 18 further comprising screening for a test compound that affects activity of the first gene.

20. A method according to claim 18 further comprising screening for a test compound that interacts with a protein encoded by the first gene.

21. A method according to claim 19 further comprising formulating into a composition a test compound that affects activity of the first gene and at least one additional component.

22. A method according to claim 1 comprising treating fish with a substance dissolved in a solvent, wherein the solvent is dimethyl sulphoxide (DMSO), methanol or ethanol.

23. (canceled)

24. A method according to claim 22 wherein the substance is dissolved in DMSO and is added to water inhabited by the fish to give a final concentration of DMSO of 1% or less.

25. A method of screening for an optimum combination of two substances, drugs, genes or drug targets that affect activity or effect of a gene of interest, or activity or effect of a treatment, or activity or effect of a physiological function or behaviour, on a fish, the method comprising:
providing, as model fish for screening, (i) fish transgenic for the gene of interest, wherein the gene of interest is under regulatory control of a specific promoter and expression of the gene of interest within the fish affects an aspect of behaviour or physiology of the fish, or (ii) fish subject to said treatment, wherein the treatment affects an aspect of behaviour or physiology of the fish;
subjecting said model fish to at least one of the steps of: (i) mutation to provide genetically mutated fish with two mutations and (ii) treatment with a test substance or two test substances to provide treated fish;
comparing an aspect of behaviour or physiology of mutated fish or treated fish with that aspect of behaviour or physiology of model fish in order to identify any mutated fish or treated fish with altered behaviour or physiology compared with model fish;
thereby to identify a combination of two substances, drugs, genes or drug targets that affects activity or effect of the gene of interest or activity or effect of said treatment, or, by identifying a genetic difference between model fish and mutated fish with such altered behaviour or physiology to identify a gene or combination of gene that affects activity or effect of the gene of interest or activity or effect of said treatment.

26. A method according to claim 25 wherein a first substance or drug is administered at an equal concentration in multiple wells, and to each individual well a further substance or drug is added, and the effect of each substance or drug in various combinations is compared to determine the optimum combination.

27. A method according to claim 26 wherein the effect of each further substance or drug in various combinations is compared to determine deleterious combinations.

28. A method according to claim 25 comprising determining the optimum combination of two potential drug targets, by comparing the effect of mutations or genetic alterations in various combinations.

29. A method according to claim 25 comprising testing combinations of at least 3, substances, drugs, genes or drug targets.

30. A method of determining whether at least two substances, drugs, genes or drug targets that affect activity or effect of a gene of interest, or activity or effect of a treatment, or activity or effect of a physiological function or behaviour, on a fish, have an additive or synergistic effect when present simultaneously, the method comprising:
providing, as model fish for screening, (i) fish transgenic for the gene of interest, wherein the gene of interest is under regulatory control of a specific promoter and expression of the gene of interest within the fish affects an aspect of behaviour or physiology of the fish, or (ii) fish subject to said treatment, wherein the treatment affects an aspect of behaviour or physiology of the fish;
subjecting said model fish to at least one of the steps of: (i) mutation to provide genetically mutated fish with two mutations and (ii) treatment with a test substance or two test substances to provide treated fish;
comparing an aspect of behaviour or physiology of mutated fish or treated fish with that aspect of behaviour or physiology of model fish in order to identify any mutated fish or treated fish with altered behaviour or physiology compared with model fish wherein a comparison is made between application of at least one of mutations and test substances individually and in combination;
thereby to determine whether a combination of at least one of (i) one or two substances, drugs, genes or drug targets that affects activity or effect of the gene of interest or activity or effect of said treatment, and (ii) one or two genetic differences between model fish and mutated fish with such altered behaviour or physiology has an additive or synergistic effect.

31. A method according to claim 30 comprising administering two substances or drugs separately and in combination

32. A method according to claim 31 wherein the effects of two substances or drugs both separately and in combination are assayed at a range of possible combinations from their respective dose-response curves when given in isolation.

33. A method according to claim 31 wherein the effect when given in combination is compared to see whether it is greater than the summation of the effect of the two substances or drugs at the same concentration given separately.

34. A method according to claim 30 comprising determining the additive or synergistic effect of two or more potential drug targets, by comparing the effect of mutations or genetic alterations in various combinations.

35. A method according to claim 30 comprising testing combinations of at least 3, substances, drugs, genes or drug targets.

36. A method of identifying a substance, drug, gene or drug target that lessens an otherwise deleterious effect or side effect of a substance, drug, gene or drug target of interest, the method comprising:
providing, as model fish for screening, (i) fish transgenic for the gene of interest, wherein the gene of interest is under regulatory control of a specific promoter and expression of the gene of interest within the fish affects an aspect of behaviour or physiology of the fish, or (ii) fish subjected to treatment with said substance of interest, wherein the treatment affects an aspect of behaviour or physiology of the fish;
subjecting said model fish to at least one of the steps of: (i) mutation to provide genetically mutated fish with at least one mutation and (ii) treatment with at least one test substance to provide treated fish;
comparing an aspect of behaviour or physiology of mutated fish or treated fish with that aspect of behaviour or physiology of model fish in order to identify any mutated fish or treated fish with altered behaviour or physiology compared with model fish; wherein comparison is optionally made between application of at least one of mutations and test substances individually and in combination;
thereby to determine whether a substance, drug, gene or drug target affects activity or effect of the substance, drug, gene or drug target of interest whereby a substance that lessens said otherwise deleterious effect or side effect is identified.

37. A method according to claim 36 wherein said substance, drug, gene or drug target of interest, has a beneficial effect, but also a negative effect in isolation, whereas in combination with the test substances, the negative effect is lessened.

38. A method according to claim 36 wherein the effect is assayed in multiwell format with a range of possible ameliorating test substances or genetic mutations.

39. A method according to claim 1 wherein a gene or mutation is identified for a patient population which is more likely to respond to a particular substance or drug, or which is less likely to respond to a particular substance or drug, or which may demonstrate a negative side effect when administered a particular substance or drug.

40. A method according to claim 39 wherein the effect of a particular mutation or polymorphism on the efficacy or side effect profile of a test substance or genetic mutation is compared with wild type response.

41. A method according to claim 40 wherein the effect is assayed in a medium or high throughput fashion in zebrafish to identify such possible polymorphisms or genetic factors resulting in variations in drug responsiveness.

42. A method according to claim 41 wherein one or more genetic factors identified in zebrafish are used to identify corresponding human genetic factors.
